(19) United States
(12) Patent Application Publication
(10) Pub. No.: US 2024/0263123 A1
(43) Pub. Date: Aug. 8, 2024

(54) SPRAY NOZZLE

(71) Applicant: Avectas Limited, Co. Kildare (IE)

(72) Inventors: Alan Patrick Pearson, Co. Kildare (IE); Shane Finnegan, Co. Kildare (IE); Simon Peter Cox, Cambridge (GB); Rishi Dhanvantray Jobanputra, Cambridge (GB); Donal Joseph Taylor, Cambridge (GB); Franck Joseph Dorien Rubiconi, Cambridge (GB)

(21) Appl. No.: 18/393,936

(22) Filed: Dec. 22, 2023

Related U.S. Application Data
(63) Continuation of application No. PCT/IB2022/055905, filed on Jun. 24, 2022.
(60) Provisional application No. 63/214,307, filed on Jun. 24, 2021.

(51) Int. Cl. C12M 1/00; G01N 1/38

(52) U.S. Cl. CPC C12M 29/06; G01N 1/38; G01N 2001/387

(57) ABSTRACT

An apparatus is provided. The apparatus can include a housing having a first end and a second end. The first end can include a sample inlet and a gas inlet. The second end can include a sample outlet and a gas outlet. The apparatus can also include a sample delivery passage extending within the housing and fluidically coupling the sample inlet to the sample outlet. The apparatus can also include a gas delivery passage extending within the housing and fluidically coupling the gas inlet to the gas outlet. Systems and methods including the apparatus are also provided herein.

### CLAIM OF PRIORITY

This application claims priority to U.S. Patent Application No. 63/214,307, filed on 24 Jun. 2021, the entire contents of which are hereby incorporated by reference.

### TECHNICAL FIELD

This disclosure describes systems, devices, and methods for a nozzle for spraying a fluid.

### BACKGROUND

Nozzles can be used in cell transfection systems to deliver fluids (e.g., atomized fluids) onto cells. Dispensing the fluids consistently under conditions maintaining the viability of the cells can be difficult using existing nozzle designs.

### SUMMARY

In one aspect, an apparatus is provided. In an embodiment, the apparatus can include a housing having a first end and a second end. The first end can include a sample inlet and a gas inlet. The second end can include a sample outlet and a gas outlet. The apparatus can also include a sample delivery passage extending within the housing and fluidically coupling the sample inlet to the sample outlet. The apparatus can also include a gas delivery passage extending within the housing and fluidically coupling the gas inlet to the gas outlet.

In another aspect, a cell-transfection spray nozzle apparatus for delivering biologically compatible aqueous-based compositions onto cells include a needle comprising a hub having a sample inlet configured for receiving a liquid sample; a proximal end portion coupled to the hub and defining a first inner diameter extending along a central axis; and a distal end portion connected to the proximal end portion and having a sample outlet for dispensing the liquid sample, the distal end portion defining a second inner diameter that is larger than the first inner diameter. The apparatus also includes a sleeve comprising: a body extending from a proximal end to a distal end, the distal end comprising a distal tip, the body defined by exterior walls and interior walls, at least a portion of the interior and exterior walls extending at an angle relative to the central axis between the proximal and distal ends, the interior walls at the proximal and distal ends being dimensioned to receive the hub and distal end portion of the needle, respectively; and four wings radially spaced from one another and extending from at least a portion of the angled exterior walls of the body. The apparatus also includes a housing configured to receive the sleeve, the housing comprising: an air inlet portion having an air inlet configured for receiving air; a first cylindrical portion fluidically coupled to the air inlet portion, the first cylindrical portion configured to releasably couple with the proximal end of the sleeve; a second cylindrical portion having an inner cylindrical diameter that is smaller than that of the first cylindrical portion, the second cylindrical portion configured to receive the distal end of the sleeve; a first conical portion fluidically coupled to and extending between the first and second cylindrical portions; a second conical portion coupled to the second cylindrical portion and having an air outlet configured for dispensing the air, the second conical portion defined by a maximum inner diameter that is equal to a minimum inner diameter of the first conical portion. The needle, the sleeve, and the housing together define cavities configured for flowing the air, the cavities comprising: a first cavity comprising the air inlet portion of the housing and an annular space defined by at least portion of the first cylindrical portion of the housing, the first conical portion of the housing, and the exterior walls of the sleeve; a plurality of second cavities that are adjacent the first cavity and defined by at least a portion of the second cylindrical portion of the housing, the wings and the angled exterior walls of the body of the sleeve; and a third cavity fluidically connected to the first cavity via the plurality of the second cavities. The third cavity extends distally in a direction along the central axis by a predetermined length that is distal to the distal tip of the sleeve and is defined by at least a portion of the exterior walls of the sleeve, the second conical portion of the housing and distal end of the needle. The second cavities define a volume that is smaller than that of the first cavity, the third cavity, or both.

In some embodiments, the sample outlet is concentrically positioned within the air outlet. In some embodiments, the four wings are equidistant from one another. In some embodiments, the needle expands to the second diameter proximal to the first conical portion. In some embodiments, the first cylindrical portion extends in a direction along the central axis, and wherein the air inlet is arranged at a right angle with respect to the first cylindrical portion. In some embodiments, the internal walls of the first and second conical portions are tapered at a same taper angle. In some embodiments, the plurality of second cavities are coplanar to one another. In some embodiments, the first diameter of the needle is at least 20% smaller than the second diameter of the needle. In some embodiments, the sleeve includes four wings spaced apart equidistant around the sleeve, the four wings are tapered to fit within the second cylindrical portion. In some embodiments, sample droplets are sprayed from the nozzle apparatus such that the sample droplets have a droplet size of about 10 to about 10.9 μm, about 11 to about 11.9 μm, about 12 to about 12.9 μm, about 13 to about 13.9 μm, about 14 to about 14.9 μm, about 15 to about 15.9 μm, about 16 to about 16.9 μm, or about 17 to about 18 μm; and wherein 80% or more of the sample droplets produced at the sample outlet have the droplet size.

In another aspect, a method of delivering atomized fluids onto cells using a cell transfection nozzle apparatus includes: introducing the liquid sample to the sample inlet of the nozzle apparatus of claim 1; introducing the air from a gas source to the gas inlet of the nozzle apparatus; flowing the gas through the cavities of the nozzle apparatus; and dispensing an atomized spray of sample droplets formed by the liquid sample exiting from the sample outlet and the gas exiting from the gas outlet of the housing.

In some embodiments, the dispensing comprises shearing the liquid sample exiting from the sample outlet with the gas exiting from the gas outlet. In some embodiments, the flowing the gas through the cavities of the nozzle apparatus comprises: flowing the gas through the first cavity; flowing the gas through the plurality of second cavities; flowing the gas through the third cavity fluidically connected to the first cavity via the plurality of the second cavities. In some embodiments, the flowing the gas through the cavities of the nozzle apparatus provides a laminar flow of gas at the third cavity, at the gas outlet, or both. In some embodiments, the first cavity subdivides the gas flowing from the gas inlet portion of the housing. In some embodiments, the method subdivides the gas into four separate passages via the plurality of second cavities that are located between the first cavity and the third cavity of the nozzle apparatus. In some embodiments, the method comprises recombining the subdivided gas in the third cavity. In some embodiments, the method includes spraying the sample droplets from the nozzle apparatus such that the sample droplets have a droplet size of about 10 to about 10.9 μm, about 11 to about 11.9 μm, about 12 to about 12.9 μm, about 13 to about 13.9 μm, about 14 to about 14.9 μm, about 15 to about 15.9 μm, about 16 to about 16.9 μm, or about 17 to about 18 μm; and wherein 80% or more of the sample droplets produced at the sample outlet have the droplet size.

In another aspect, an apparatus includes: a housing including a first end and a second end, the first end including a sample inlet and a gas inlet, and the second end including a sample outlet and a gas outlet; a sample delivery passage extending within the housing and fluidically coupling the sample inlet to the sample outlet; and a gas delivery passage extending within the housing and fluidically coupling the gas inlet to the gas outlet.

In another aspect, a method includes: coupling a sample source including a sample to a sample inlet arranged at an first end of a housing of a nozzle; coupling a gas source including a gas to a gas inlet arranged at the first end of the housing of the nozzle; providing the sample within a sample delivery passage extending within the housing and fluidically coupling the sample inlet to a sample outlet arranged at an output orifice at a second end of the housing of the nozzle; and providing the gas within a gas delivery passage extending within the housing and fluidically coupling the gas inlet to a gas outlet arranged at the output orifice at the second end of the housing of the nozzle; wherein the gas and the sample are dispensed from the output orifice as an atomized spray to cells arranged on a filter membrane of a cell transfection system.

In another aspect, a nozzle apparatus includes a needle, a sleeve, and a housing configured to receive the sleeve. The needle includes a hub having a sample inlet configured for receiving a liquid sample; a proximal end portion coupled to the hub and defining a first inner diameter extending along a central axis; and a distal end portion connected to the proximal end portion and having a sample outlet for dispensing the liquid sample. The sleeve includes a body extending from a proximal end to a distal end, the distal end comprising a distal tip, the body defined by exterior walls and interior walls; and a plurality of wings radially spaced from one another and extending from the exterior walls of the body. The housing includes a gas inlet portion having a gas inlet configured for receiving a gas; a first portion fluidically coupled to the gas inlet portion; a second portion configured to receive the distal end of the sleeve; a third portion that is a conical in shape and fluidically coupled to and extending between the first and second portions; and a fourth portion that is conical in shape and coupled to the second portion and having a gas outlet configured for dispensing the gas. The housing, the sleeve, and needle together define cavities configured for flowing the gas, the cavities including: a first cavity comprising the gas inlet portion and an annular space defined by at least a portion of the first and third portions of the housing and the exterior walls of the sleeve; a plurality of second cavities that are adjacent the first cavity and defined by at least a portion of the second portion of the housing, the wings of the sleeve, and the exterior walls of the body of the sleeve; and a third cavity fluidically connected to the first cavity via the plurality of the second cavities, wherein the third cavity extends distally in a direction along the central axis by a predetermined length that is distal to the distal tip of the sleeve; and the third cavity is defined by at least a portion of the exterior walls of the sleeve, the third portion of the housing and distal end of the needle.

In some embodiments, the apparatus can include an output orifice at the second end of the housing. The output orifice can be configured to output a spray (e.g., an atomized spray) comprising a gas and a sample from the apparatus. In some embodiments, the output orifice can include the gas outlet and the sample outlet. In some embodiments, the gas delivery passage can include a tapered portion at the second end of the housing. The tapered portion can include a taper angle measured between a longitudinal axis of the gas delivery passage and a longitudinal axis of the gas outlet. In some embodiments, taper angle can be between 0-4.9 degrees, 5-10 degrees, 11-15 degrees, 16-20 degrees, 21-25 degrees, 26-30 degrees, 31-35 degrees, 36-40, 41-45 degrees, or 46-50 degrees.

In some embodiments, the gas outlet can be annularly-shaped and the sample outlet can be disposed within the annularly-shaped gas outlet. In some embodiments, the gas inlet can be fluidically coupled to a plurality of gas delivery passages in the housing. Each gas delivery passage can be fluidically coupled to a respective gas outlet included in a plurality of gas outlets. In some embodiments, the plurality of gas outlets can be arranged circumferentially around the sample outlet. In some embodiments, the plurality of gas outlets can be circular-shaped, or semi-circular-shaped.

In some embodiments, the housing can further include a mixing chamber fluidically coupled to the gas outlet and to the sample outlet. The mixing chamber can be fluidically coupled to the output orifice via an output passage. In some embodiments, a diameter of the sample delivery passage can be 0.6-0.79 mm, 0.8-0.89 mm, 0.9-0.99 mm, 1.0-1.09 mm, 1.10-1.19 mm, or 1.20-1.30 mm. In some embodiments, a diameter of the gas delivery passage can be 0.6-0.79 mm, 0.8-0.89 mm, 0.9-0.99 mm, 1.0-1.09 mm, 1.10-1.19 mm, or 1.20-1.30 mm. In some embodiments, the apparatus can include a photopolymer material.

In another aspect, a method is provided. In an embodiment, the method can include coupling a sample source including a sample to a sample inlet arranged at a first end of a housing of a nozzle. The method can also include coupling a gas source including a gas to a gas inlet arranged at the first end of the housing of the nozzle. The method can further include providing the sample within a sample delivery passage extending within the housing and fluidically coupling the sample inlet to a sample outlet arranged at an output orifice at a second end of the housing of the nozzle. The method can also include providing the gas within a gas delivery passage extending within the housing and fluidically coupling the gas inlet to a gas outlet arranged at the output orifice at the second end of the housing of the nozzle; wherein the gas and the sample are dispensed from the output orifice as a spray (e.g., atomized spray) to cells arranged on a filter membrane of a cell transfection system.

In some embodiments, the sample can be provided at a pressure between 100-119 mbar, 120-129 mbar, 130-139 mbar, 140-149 mbar, 150-199 mbar, 200-249 mbar, 250-349 mbar, 350-399 mbar, 400-449 mbar, 450-499 mbar, 500-549 mbar, 550-599 mbar, 600-649 mbar, 650-699 mbar, or 700-750 mbar. In some embodiments, gas can be provided at a flow rate between 5-9.9 LPM, 10-14.9 LPM, 15-19.9 LPM, 20-24.9 LPM, or 25-30 LPM. In some embodiments, the spray (e.g., atomized spray) can be provided at a cone angle between 20-21.9 degrees, 22-23.9 degrees, 24-25.9 degrees, 26-27.9 degrees, or 28-30 degrees. In some embodiments, the spray can include sample droplets having a droplet size between 10-10.9 μm, 11-11.9 μm, 12-12.9 μm, 13-13.9 μm, 14-14.9 μm, 15-15.9 μm, 16-16.9 μm, or 17-18 μm; and 80% or more of the sample droplets produced by the output orifice can have the droplet size. In some embodiments, the spray can provide a dosage of sample to the cells between 40-44.9 mg, 45-49.9 mg, 50-54.9 mg, 55-59.9 mg, or 60-64.9 mg. In some embodiments, the spray can contact the cells at an impact pressure between 15-19.9 Pa, 20-24.9 Pa, 25-29.9 Pa, 30-34.9 Pa, 35-39.9 Pa, 40-44.9 Pa, 45-49.9 Pa, 50-54.9 Pa, or 55-59.9 Pa.

In another aspect a method is provided. In an embodiment, the method can include receiving a mold for a nozzle including a first end and a second end, a housing, a sample inlet, a sample outlet, a gas inlet, a gas outlet, a sample delivery passage extending within the housing and fluidically coupling the sample inlet to the sample outlet, a gas delivery passage extending within the housing and fluidically coupling the gas inlet to the gas outlet, and an output orifice at the second end of the nozzle. The output orifice can include the sample outlet and the gas outlet. The method can also include forming the nozzle from the mold.

In some embodiments, the nozzle can include a tapered portion at the second end of the nozzle. The tapered portion can include a taper angle measured between a longitudinal axis of the gas delivery passage and a longitudinal axis of the gas outlet. In some embodiments, the nozzle is formed by injection molding. In some embodiments, the nozzle is formed from a photopolymer. In some embodiments, the nozzle is formed from a biocompatible material.

In another aspect, a system is provided. In an embodiment, the system can include a housing configured to receive a filter plate comprising a well. The system can also include a differential pressure applicator configured to apply a differential pressure to the well. The system can further include a nozzle configured to deliver an atomized delivery solution to the well. The system can also include a culture medium applicator configured to deliver a culture medium to the well. The nozzle can include a housing including a first end and a second end. The first can include a sample inlet and a gas inlet and the second end can include a sample outlet and a gas outlet. The nozzle can also include a sample delivery passage extending within the housing and fluidically coupling the sample inlet to a sample outlet. The nozzle can further include a gas delivery passage extending within the housing and fluidically coupling the gas inlet to a gas outlet.

In some embodiments, the nozzle can include an output orifice at the second end of the housing. The output orifice can output a spray (e.g., an atomized spray) comprising a gas and a sample from the apparatus. In some embodiments, the output orifice can include the gas outlet and the sample outlet. In some embodiments, the housing can include a mixing chamber fluidically coupled to the gas outlet and to the sample outlet. The mixing chamber can be fluidically coupled to the output orifice via an output passage.

The details of one or more implementations are set forth in the accompanying drawings and the description below. Other features, objects, and advantages will be apparent from the description and drawings, and from the claims.

### DESCRIPTION OF DRAWINGS

FIG. 1A illustrates a cross-sectional view of an exemplary implementation of a nozzle for use in a cell transfection system.

FIG. 1B illustrates a cross-sectional view of the nozzle of FIG. 1A taken from the perspective of section A-A.

FIG. 1C illustrates a cross-sectional view of detail B of the nozzle of FIG. 1A.

FIG. 1D illustrates a cross-sectional view of detail C of the nozzle of FIG. 1B.

FIG. 2A illustrates a cross-sectional view of the nozzle of FIGS. 1A-1D.

FIG. 2B illustrates a cross-sectional view of an output orifice of the nozzle of FIGS. 1A-1D.

FIG. 2C is a plot of sizes of droplets dispensed from the nozzle of FIGS. 1A-1D.

FIG. 2D is an image of a spray dispensed from the nozzle of FIGS. 1A-1D.

FIG. 2E is another image of the spray dispensed from the nozzle of FIGS. 1A-1D.

FIG. 2F is a plot of core spray angle and deflection to device axis of the spray dispenses from the nozzle of FIGS. 1A-1D.

FIG. 2G is an image of a spray pattern dispensed from the nozzle of FIGS. 1A-1D.

FIG. 2H is a plot of a mass of material in the spray dispensed from the nozzle of FIGS. 1A-1D.

FIG. 3 includes images of spray patterns dispensed at various air flow rates from the nozzle of FIGS. 1A-1D.

FIG. 4 is a plot of sizes of droplets dispensed from the nozzle of FIGS. 1A-1D at various air flow rates.

FIG. 5 is a plot of cone angle and deflection angle of a spray dispensed from the nozzle of FIGS. 1A-1D at various air flow rates.

FIG. 6A illustrates an isometric cross-sectional view of an exemplary implementation of the nozzle of FIGS. 1A-1D.

FIG. 6B illustrates an isometric view of an input end of the nozzle of FIG. 6A.

FIG. 6C illustrates an isometric view of an output end of the nozzle of FIG. 6A.

FIG. 6D illustrates a perspective view of a sleeve of the nozzle of FIGS. 6A-6I.

FIG. 6E is a bottom view of the sleeve of FIG. 6D.

FIG. 6F is a cross-sectional view along section line 6F of FIG. 6A.

FIG. 6G is a cross-sectional view along section line 6G of FIG. 6A.

FIG. 6H is a cross-sectional view along section line 6H of FIG. 6A.

FIG. 6I is a cross-sectional view along section line 6I of FIG. 6A.

FIG. 7A illustrates an cross sectional view of a plurality of cavities of the nozzle of FIGS. 6A-6I.

FIG. 7B illustrates a perspective cross-sectional view of the cavities of FIG. 7A, removed from the nozzle.

FIG. 7C illustrates an exploded view of the cavities of FIGS. 7A and 7B.

FIG. 7D illustrates an exploded view of the nozzle of FIGS. 6A-6I.

FIG. 8A illustrates an isometric view of an exemplary implementation of a needle of the nozzle of FIGS. 6A-7.

FIG. 8B illustrates a cross-sectional view of the needle of FIG. 8A.

FIG. 9A illustrates a cross-sectional view of another exemplary implementation of a nozzle for use in a cell transfection system.

FIG. 9B illustrates a cross-sectional view of the nozzle of FIG. 9A taken from the perspective of section A-A.

FIG. 9C illustrates a cross-sectional view of detail B of the nozzle of FIG. 9A.

FIG. 9D illustrates a cross-sectional view of detail C of the nozzle of FIG. 9B.

FIG. 10A illustrates a cross-sectional view of the nozzle of FIGS. 9A-9D.

FIG. 10B is a plot of sizes of droplets dispensed from the nozzle of FIGS. 9A-9D.

FIG. 10C is an image of a spray dispensed from the nozzle of FIGS. 9A-9D.

FIG. 10D is another image of the spray dispensed from the nozzle of FIGS. 9A-9D.

FIG. 10E is a plot of core spray angle and deflection to device axis of the spray dispenses from the nozzle of FIGS. 9A-9D.

FIG. 10F is an image of a spray pattern dispensed from the nozzle of FIGS. 9A-9D.

FIG. 10G is a plot of a mass of material in the spray dispensed from the nozzle of FIGS. 9A-9D.

FIG. 11 includes images of spray patterns dispensed at various flow rates from the nozzle of FIGS. 9A-9D.

FIG. 12 is a plot of sizes of droplets dispensed from the nozzle of FIGS. 9A-9D at various air flow rates.

FIG. 13 is a plot of cone angle and deflection angle of a spray dispensed from the nozzle of FIGS. 9A-9D at various air flow rates.

FIG. 14A illustrates an isometric cross-sectional view of an exemplary implementation of the nozzle of FIGS. 9A-9D.

FIG. 14B illustrates an isometric view of an input end of the nozzle of FIG. 14A.

FIG. 14C illustrates an exploded view of the nozzle of FIG. 14A.

FIG. 15 is a plot of flow rates at various input pressures and operating points by the nozzle of FIGS. 1A-1D.

FIG. 16 is a plot of sizes of droplets dispensed from exemplary implementations of the nozzle of FIGS. 1A-1D.

FIG. 17 is a plot of flow rates at various input pressures and operating points by the nozzle of FIGS. 9A-9D.

FIG. 18 is a plot of sizes of droplets dispensed from exemplary implementations of the nozzle of FIGS. 9A-9D.

FIG. 19 is an interpolation plot illustrating relationships between sizes of droplets dispensed from the nozzles of FIGS. 1A-1D and 9A-9D for various air flow rates and manufacturing materials.

FIG. 20 is a process flow diagram illustrating an exemplary process for using the nozzle of FIGS. 1A-1D or 9A-9D in a cell transfection system.

FIG. 21 is a process flow diagram illustrating an exemplary process for manufacturing the nozzle of FIGS. 1A-1D or 9A-9D in cell transfection system.

FIG. 22 is an isometric view of a computer aided design (CAD) drawing illustrating an example embodiment of a cell transfection system according to some embodiments disclosed herein.

FIG. 23A is a side view of the cell transfection system shown in FIG. 22.

FIG. 23B is a front view of the cell transfection system shown in FIG. 22.

FIG. 24 is a side view of another example embodiment of the cell transfection system shown in FIG. 22, according to some embodiments discloses herein.

FIG. 25 illustrates an image of another example embodiment of a cell transfection system according to some embodiments disclosed herein.

FIG. 26 illustrates a view of the cell transfection system shown in FIG. 25.

FIG. 27 illustrates a second view of the cell transfection system shown in FIG. 25.

FIG. 28 illustrates a close-up view of a portion of the cell transfection system shown in FIG. 25.

FIG. 29 is a table showing additional embodiments of an implementation of the nozzle of FIGS. 1A-1D.

FIG. 30 is a table showing additional embodiments of another implementation of the nozzle of FIGS. 1A-1D.

FIG. 31 is a table showing additional embodiments of an implementation of the nozzle of FIGS. 9A-9D.

Like reference symbols in the various drawings indicate like elements.

### DETAILED DESCRIPTION

Cell transfection can include applying a fluid solution to the surface of cells for transfection. The fluid can be provided as a spray (e.g., an atomized spray) to the cells to improve delivery of materials to the cells. The spray can be dispensed onto the cells by a nozzle configured in a cell transfection system. The nozzle can receive the fluid solution a gas and can mix the fluid solution with the gas to form the spray.

Commercial atomization nozzles are generally intended to be run continuously or semi-continuously for seconds, minutes, hours or days. Performance can be characterized at steady-state with no emphasis on atomization performance at commencement of atomization or the cessation of atomization. Examples of such nozzles can include metered dose inhalers, nozzles for nebulization of drugs, and tablet coating nozzles. The aforementioned devices can be configured operating durations ranging from seconds to days. In addition, metered dose inhalers can include a nozzle designed for cargoes including small molecule, peptide, protein, nucleic acid delivery, however these nozzles can create high shearing forces on the cargo molecule to be delivered. As such, a nozzle as used in a metered dose inhaler may not be substitutable or enabled for the nozzle and systems described herein.

Maintaining cell viability and effectively delivering materials to cells for cell transfection can be an important consideration in the design of components of cell transfection systems. It can be challenging to design nozzles to deliver sprays (e.g., atomized sprays) of fluids such that adequate delivery can be achieved and cell viability can be maintained. For example, a nozzle design dispensing too much of a solution, such as a solution containing a permeabilizing agent, can reduce cell viability. A nozzle design dispensing too little permeabilization solution may not adequately permeabilize the cells.

Some implementations of the nozzles described herein can allow variations in pressure between 1600 mbar and 3000 mbar. Resulting changes in velocity can occur without resulting in changes in the symmetry, uniformity and consistency of the spray pattern. As a result, some implementations of the nozzles described herein can provide various degrees of freedom with which to optimise or tune transfection processes for particular target cell types. Some implementations of the nozzles described herein can produce droplets similar in size to the size of target cells (e.g., approximately 10 micrometers) so that a 1:1 ratio of droplet size to cell size can be achieved, which can provide for improved and/or optimal transfection conditions. The nozzles and/or a spray nozzles can include an atomizer in any of the embodiments described herein.

The nozzle apparatuses and nozzle systems described herein can be configured as single use apparatuses or single use systems where the nozzle apparatus and/or the nozzle systems are disposable. In some aspects, the nozzle apparatus and/or the nozzle systems are configured for multiple uses where the nozzle(s) can be cleaned, sterilizes, washed out, or flushed between uses.

Similarly, the impact pressure at which the fluid or material contacts the cells can affect delivery to the cell. For example, droplets impacting with cells with sufficient force, such as droplets dispersed at particular pressures can perturb the membranes of the cells. If the spray of fluid contacts the cells at too high of an impact pressure, the cells may be damaged and become unviable for transfection. If the spray of the fluid contacts the cells at a low impact pressure, delivery of materials to cells may be reduced.

Additionally, the pattern of the spray can also affect the delivery of materials to the cells. If the spray pattern is irregularly shaped or not provided directly to a surface on which the cells are located, delivery can be reduced.

Thus, delivery and cell viability can be affected by a variety of factors related to nozzle design. Providing a consistent spray with a flow rate, impact pressure, droplet size, and spray pattern to effectively deliver materials to cells can be difficult to achieve using conventional nozzle designs.

Some implementations of the current subject matter can include a nozzle for use in delivery of materials to cells, such as for cell transfection. Some implementations of the nozzle described herein can include fluid and gas delivery passages that can include respective outlets arranged to provide mixing of gas and fluid to form a spray (e.g., an atomized spray), which in some implementations can enhance delivery of materials to cells and maintain cell viability. For example, in some embodiments, the gas outlet can be configured in a concentric arrangement with respect to the sample outlet so that a deviation of the spray from a central axis can be reduced as compared to existing nozzle designs. High fluidic resistance in the sample line of the nozzle can allow for the use of higher sample pressures to deliver the required dose. Higher pressures can be beneficial as they are usually more within a mid-range of pressure regulating devices, rather than at a lower end of the pressure regulating devices, which can improve overall dose accuracy.

The use of standard fluidic connections can enable easier manufacture of the nozzle compared to some existing nozzle designs. Some embodiments described here and their geometric features can be tuned to optimize the spray so that features such as droplet size, cone angle, and impact force can be readily achieved using pressure controllers. The modular format of the embodiments described herein can also enable easier manufacture of complex internal geometries within the nozzle.

Some implementations of the nozzle and nozzle designs described herein can provide a more precisely directed spray pattern onto the cells at a variety of fluid and gas flow rates more consistently than current nozzles. In some implementations, the nozzle described herein can improve the impact pressure and droplet size of the delivery fluid so as to improve cell delivery and maintain cell viability. A demonstrated advantage of the nozzle designs described herein is the reduction in damage to biological cargos typically used in cell engineering. As such, some implementations of the nozzle design described herein can cause little to no shear stress related damage to target cells.

In addition, some implementations of the nozzle described herein can commence and cease atomization approximately instantaneously (e.g., very quickly) and can run at operational steady state for durations of milliseconds to seconds. For successful transfection of cells on a filter membrane opposing the nozzle, the spray or plume of droplets (e.g., atomized droplets) needs to be symmetrical, uniform, consistently reproducible, and have minimal transition times between an “off” configuration and an “on” configuration and vice versa. Some implementations of the nozzles described herein can achieve transition times for transitions between “off” and “on” configurations can be less than 20 ms, while transition times for transitions between “on” and “off” configurations can be less than 10 ms. The lack of large transition times between “on” and “off” configurations (and vice versa) can enable some implementations of the nozzles described herein to provide more predictable and efficient sprays on to the target cells for shorter overall transfection durations and thus make transfection more efficient. Existing cell transfection systems can require transfection durations over 500 ms, which can cause a greater degree of perturbation of the cells and decreased viability of the cells.

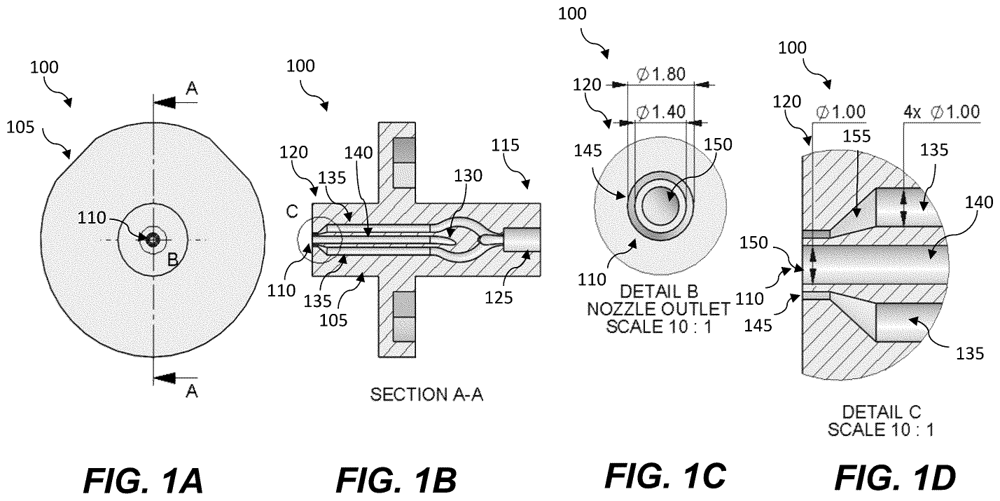

FIG. 1A illustrates a cross-sectional view of an exemplary implementation of a nozzle for use in a cell transfection system. As shown in FIG. 1A, nozzle 100 includes a housing 105 and an output orifice 110. In some embodiments, the housing 105 can be cylindrical shaped, although other shapes can be included. The output orifice 110 can be configured to dispense a spray (e.g., atomized spray) formed from a fluid and a gas that are mixed by the nozzle 100.

FIG. 1B illustrates a cross-sectional view of the nozzle of FIG. 1A taken from the perspective of section A-A. As shown in FIG. 1B, the nozzle 100 can include an input end 115 at which a gas supply including a gas can be coupled to the nozzle 100. In some embodiments, a fluid supply including a fluid can also be coupled to the nozzle 100 at the input end 115. In some embodiments, the fluid can be a sample that is included in a sample supply coupled to the nozzle 100 at the input end 115. The nozzle 100 can also include an output end 120. The nozzle 100 can also include an output end 120 at which a spray including the gas and the fluid or sample can be dispensed. The housing 105 can similarly include an input end and an output end, which can respectively correspond to the input end 115 and the output end 120.

The nozzle 100 can include a gas inlet 125 arranged at the input end 115. The gas inlet 125 can be coupled to a gas supply and can be fluidically coupled to one or more gas delivery passages 135. The gas delivery passages 135 can extend within the housing 105 and can terminate at the output end 120. The nozzle 100 can also include a fluid or sample inlet 130 arranged at the input end 115. The fluid or sample inlet 130 can be fluidically coupled to one or more sample delivery passages 140. The sample delivery passage 140 can extend within the housing 105 and can terminate at the output end 120.

In some embodiments, the sample delivery passage 140 can be cylindrical and can be configured axially through the nozzle 100 without any internal features, or without changes in the internal diameter of the nozzle. This simple fluidic pathway can minimize turbulent flow and formation of bubbles within the nozzle 100, which can contribute to achieving a uniform and consistent atomization spray or plume of droplets (e.g., atomized droplets). The internal volume of the central fluidic pathway can be approximately 15 uL.

FIG. 1C illustrates a cross-sectional view of detail B of the nozzle of FIG. 1A. As shown in FIG. 1C, the output end 120 can include the output orifice 110 and can also include a gas outlet 145 and a sample outlet 150. In some embodiments, the nozzle 100 can include one or more gas outlets 145 and one or more sample outlets 150. In some embodiments, the output orifice 110 can include one or more gas outlets 145 and one or more sample outlets 150. The nozzle 100 can be configured to mix the gas and the fluid external from the output orifice 110.

The gas outlet 145 can be is concentric to the sample outlet 150 and can create a straighter flow path for the atomizing gas when ejected from the nozzle. The small diameter feature of the concentric gas outlet 145 can provide a uniform or optimal shear stress on the sample when atomizing. The sample outlet 150 can be parallel to the gas outlet 145 which can allow for a well formed and fluidically stable injection of liquid into the nozzle air flow. The overall geometric features of the nozzle described herein can allow for the exploration of a range of operating conditions while maintaining a symmetric spray and minimizing yield loss during spraying.

FIG. 1D illustrates a cross-sectional view of detail C of the nozzle of FIG. 1B. As shown in FIG. 1D, the gas delivery passage 135 can be coupled to the gas outlet 145 via a tapered portion 155. The tapered portion 155 can be configured with a taper angle that is measured relative to a central axis of the gas delivery passage 135 and the gas outlet 145.

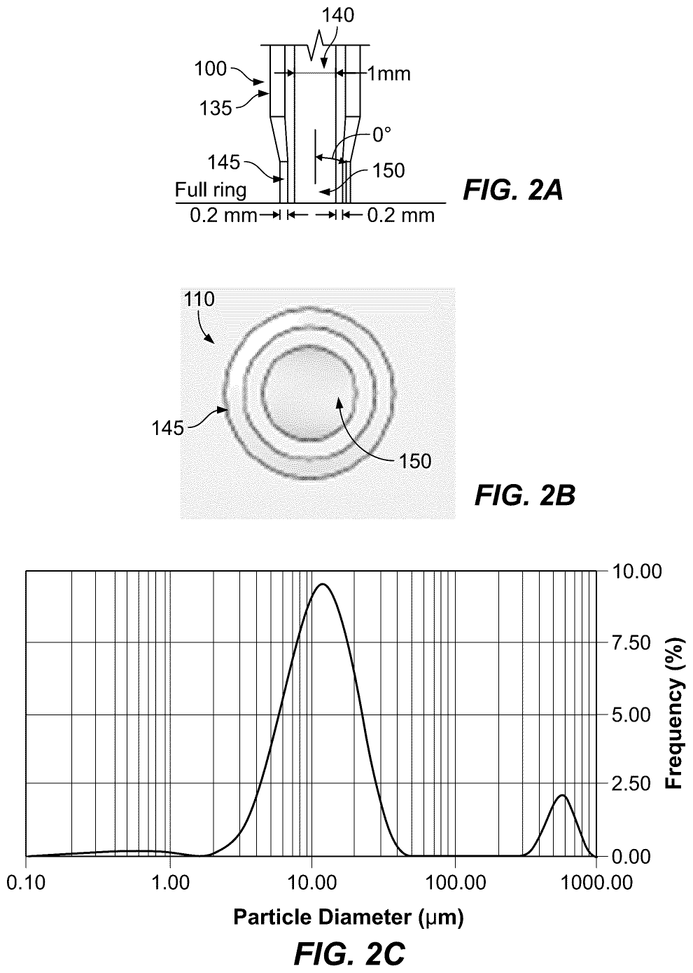

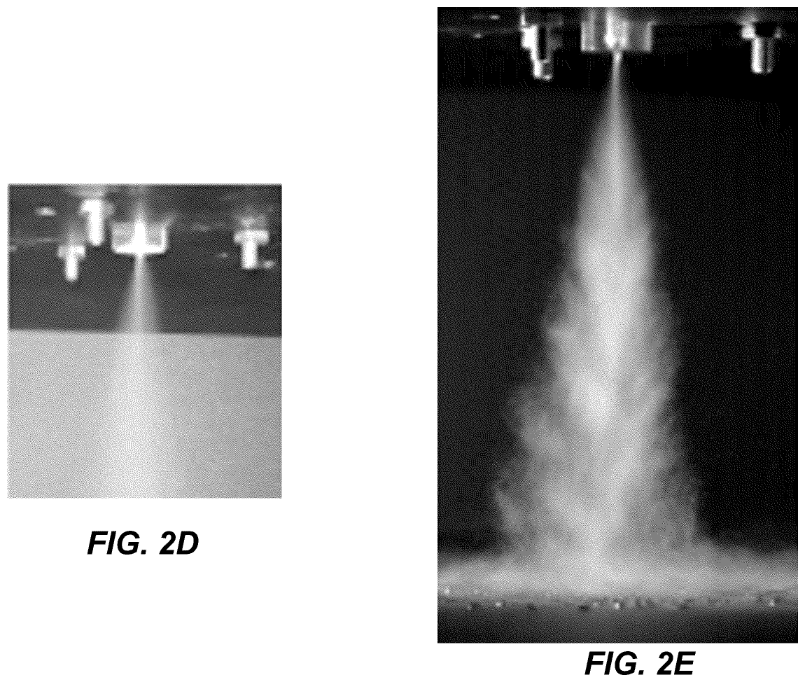

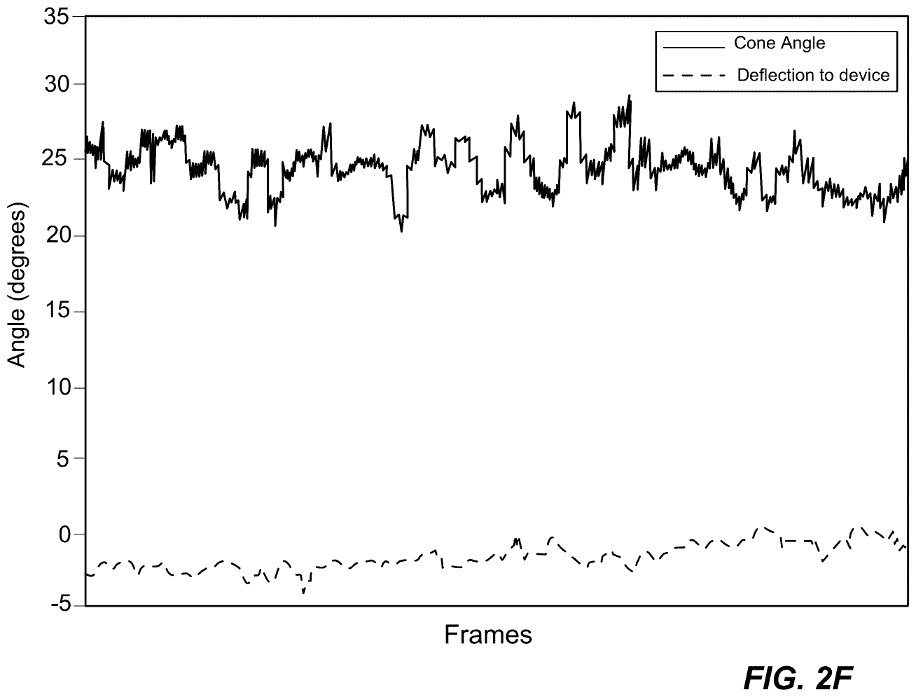

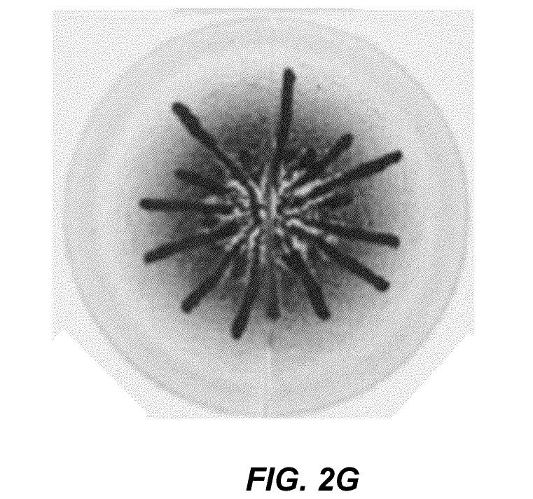

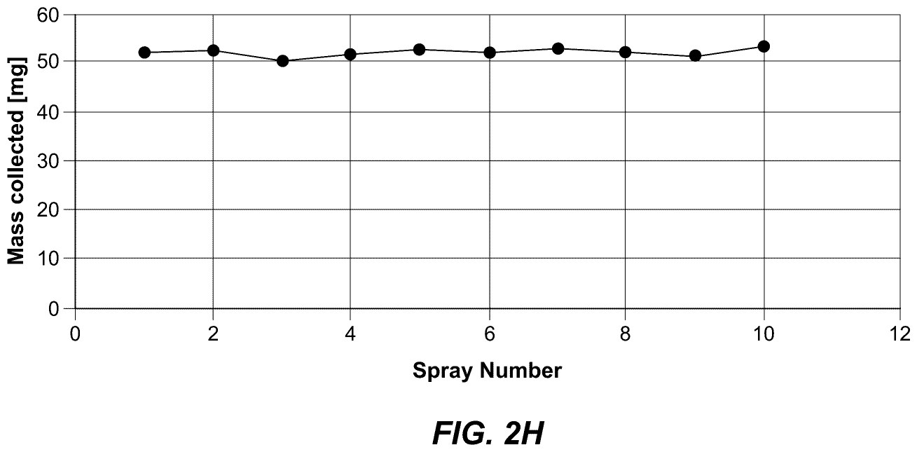

FIG. 2A illustrates a cross-sectional view of the nozzle of FIGS. 1A-1D. As shown in FIG. 2A, in some embodiments, the nozzle 100 can include a sample delivery passage 140 having a diameter of 1 mm. In some embodiments, the diameter of the sample delivery passage 140 can be the same or different than the diameter of the sample outlet 150. In some embodiments, the diameter of the sample delivery passage 140 (and/or the sample outlet 150) can be between 0.6-0.79 mm, 0.8-0.89 mm, 0.9-0.99 mm, 1.0-1.09 mm, 1.10-1.19 mm, or 1.20-1.30 mm. In some implementations, the diameter of the sample delivery passage 140 and/or the sample outlet 150 can be less than 0.6 mm, or greater than 1.3 mm. The diameter of the sample delivery passage 140 and/or the sample outlet 150 can be selected based on a surface tension of the sample or a pressure at which the sample is provided to the nozzle.

As further shown in FIG. 2A, the nozzle 100 can include a gas delivery passage 135 and a gas outlet 145 arranged around the sample delivery passage 140 and the sample outlet 150, respectively. In some embodiments, the diameter of the gas delivery passage 135 (and/or the gas outlet 145) can be 0.6-0.79 mm, 0.8-0.89 mm, 0.9-0.99 mm, 1.0-1.09 mm, 1.10-1.19 mm, or 1.20-1.30 mm. In some implementations, the diameter of the gas delivery passage 135 and/or the gas outlet 145 can be less than 0.6 mm, or greater than 1.3 mm. As shown in FIG. 2A, the diameter of the example gas outlet 145 is 0.2 mm.

A taper angle, θ, can be formed between a longitudinal or central axis of the gas outlet 145 and a longitudinal or central axis of the gas delivery passage 135. In some embodiments, the taper angle θ can be between 0-4.9 degrees, 5-10 degrees, 11-15 degrees, 16-20 degrees, 21-25 degrees, 26-30 degrees, 31-35 degrees, 36-40, 41-45 degrees, or 46-50 degrees. In some embodiments, the taper angle can be greater than 50 degrees.

FIG. 2B illustrates a cross-sectional view of an output orifice 110 of the nozzle of FIGS. 1A-1D. As shown in FIG. 2B, the gas outlet 145 has an annular shape and is located around the outer circumference of the sample outlet 150. The annular shape of the gas outlet 145 can allows for atomization at the tip of the nozzle using symmetric air outlets with the intent to make the air stream converge towards the sample outlet 150 while maximizing the applied shear on the liquid bulk of the sample. This can deliver a symmetrical and stable spray.

FIG. 2C is a plot of sizes of droplets dispensed from the nozzle of FIGS. 1A-1D. As shown in FIG. 2C, the size of the droplets dispensed from the nozzle 100 can be about 10-12 μm. In some embodiments, the size of the droplets can be between 0-9.9 μm, 10-10.9 μm, 11-11.9 μm, 12-12.9 μm, 13-13.9 μm, 14-14.9 μm, 15-15.9 μm, 16-16.9 μm, or 17-18 μm. In some embodiments, the droplets can be larger than 18.0 μm. Uniform droplet size distribution can allow uniform distribution of cargo. In addition, the droplet size range can be similar to the size range of target cells, which has been shown to result in good transfection of cells. In some embodiments, 80% or more of the sample droplets produced by the output orifice 110 can have droplet sizes in the ranges described above.

FIG. 2D is an image of a spray dispensed from the nozzle of FIGS. 1A-1D. As shown in the image of FIG. 2D, the spray is well developed and atomized to form a symmetric pattern or shape, which can advantageously result in uniform distribution of the atomized sample (or cargo) across the target surface of cells.

FIG. 2E is another image of the spray dispensed from the nozzle of FIGS. 1A-1D. As shown in FIG. 2E, the cone of spray emitted from nozzle 100 is perpendicular to the surface below the nozzle 100 and is minimally deflected relative to a central axis of the nozzle 100. In this way, the cone of spray can consistently cover a surface on which cells are located for delivery without requiring adjustment or additional equipment to correct for deflection angles of the cone of spray.

FIG. 2F is a plot of core spray angle and deflection to device axis of the spray dispenses from the nozzle of FIGS. 1A-1D. As shown in FIG. 2F, the cone angle (upper plot line) can vary between 20 and 30 degrees. The cone angle can be measured from the center axis of the cone to an outer edge of the cone measured at the surface that the cone of spray is contacting. In some embodiments, the cone angle can be between 20-21.9 degrees, 22-23.9 degrees, 24-25.9 degrees, 26-27.9 degrees, or 28-30 degrees. The angle of the deflection relative to the nozzle 100 (lower plot line) can vary between 0 and-5 degrees.

FIG. 2G is an image of a spray pattern dispensed from the nozzle of FIGS. 1A-1D. As shown in FIG. 2G, the spray pattern emitted by nozzle 100 can be uniform distribution with concentrations of droplets that are radially arranged. As shown in the image of FIG. 2G, the atomized cargo is evenly distributed. The radial pattern may suggest a secondary mode of action for transfection as the sample can flow out along the radial lines and into contact with the cells.

FIG. 2H is a plot of a mass of material in the spray dispensed from the nozzle of FIGS. 1A-1D. The material can be the sample or the fluid that is provided within the spray. As shown in FIG. 2H, the mass (or dosage) of material provided in the atomized spray of nozzle 100 can vary between 50 and 60 mg. In some embodiments, the mass or dosage can be between 40-44.9 mg, 45-49.9 mg, 50-54.9 mg, 55-59.9 mg, or 60-64.9 mg. The material or sample can be provided onto the cells with an impact pressure between 15-19.9 Pa, 20-24.9 Pa, 25-29.9 Pa, 30-34.9 Pa, 35-39.9 Pa, 40-44.9 Pa, 45-49.9 Pa, 50-54.9 Pa, 55-59.9 Pa, or greater than 59.9 Pa. In some embodiments, the impact pressure can be less than 15.0 Pa or greater than 60.0 Pa.

Exercising tight control over the mass of sample material delivered can be important for dose control in cell transfection systems. Appropriate dose control can ensure transfection occurs at an optimum level, but can also reduce the potential for cell damage due to over exposure to cargo. Similarly, controlling the impact pressure can protect cells form excessive damage while providing enough pressure to facilitate membrane perturbation.

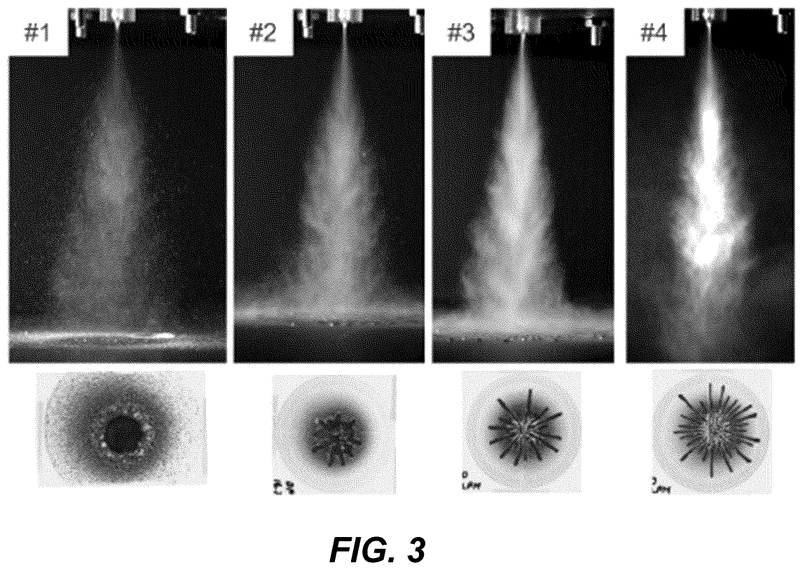

FIG. 3 includes images of spray patterns dispensed at various flow rates from the nozzle of FIGS. 1A-1D. As shown in FIG. 3, the nozzle 100 can emit different shaped sprays at different flow rates. For example, the settings in table 1 correspond to cone images 1-4 and the related spray pattern images shown below each cone image.

TABLE 1

| Setting | #1 | | #2 | | #3 | | #4 | |
|---|---|---|---|---|---|---|---|---|
| Air Pressure | 600 | mbar | 1300 | mbar | 1980 | mbar | 2975 | mbar |
| Air flow rate | 10 | LPM | 16.7 | LPM | 21.9 | LPM | 29.3 | LPM |
| Impact | 11 | Pa | 27 | Pa | 42.3 | Pa | 69.9 | Pa |
| Pressure | | | | | | | | |

As shown in FIG. 3, the spray cone can change shape based on the pressure of the supplied air. The structure of the nozzle described herein can allow the spray to be tuned so that a balance between impact pressure and droplet distribution can be optimized for the cell type being targeted.

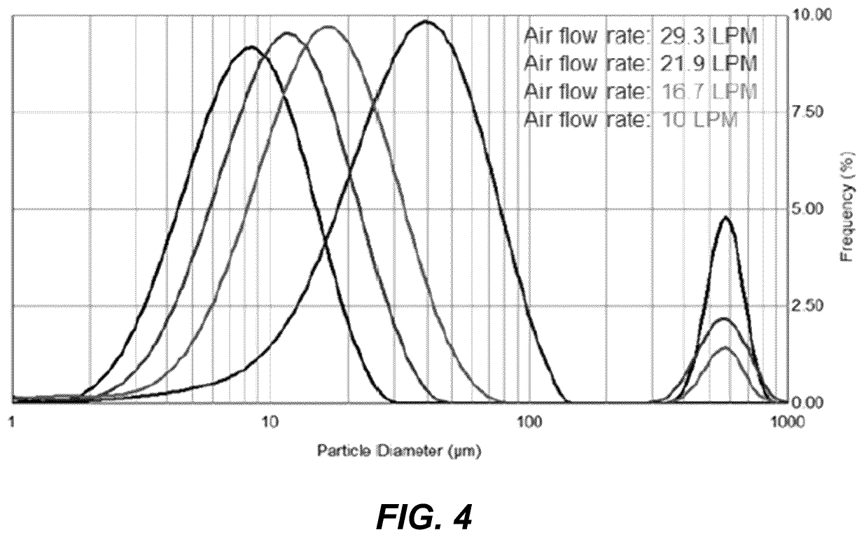

FIG. 4 is a plot of sizes of droplets dispensed from the nozzle of FIGS. 1A-1D at various flow rates. As shown in FIG. 4, the nozzle 100 can dispense droplets that have different sizes when the fluid or spray is dispensed at different flow rates. For example, as shown in FIG. 4, at lower flow rates the droplet size is larger. At faster flow rates, the droplet size is smaller. In this way, the nozzle described herein can allow tuning of the spray based on the cell type of interest.

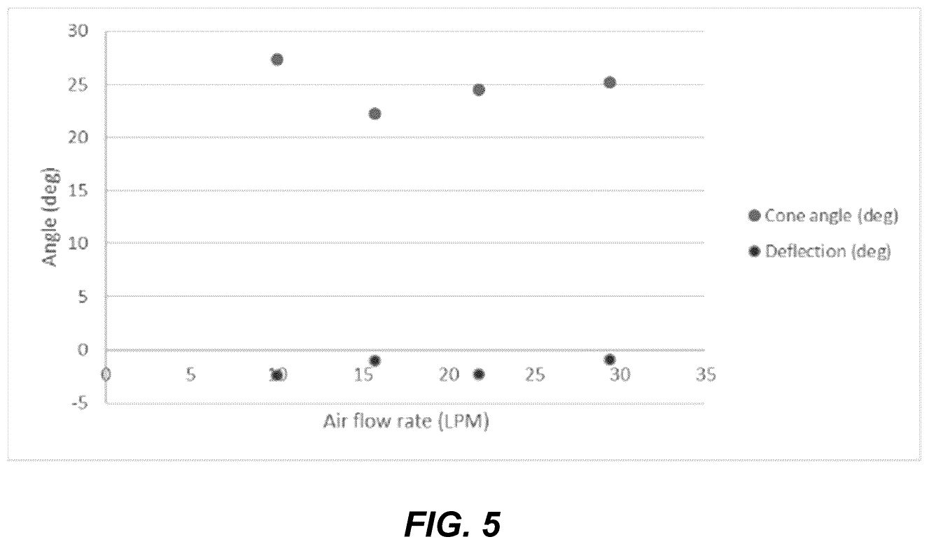

FIG. 5 is a plot of cone angle and deflection angle of a spray dispensed from the nozzle of FIGS. 1A-1D at various air flow rates. As shown in FIG. 5, the cone angle of the spray emitted from nozzle 100 can vary between 20 and 30 degrees for air flow rates between 0 and 35 LPM. The deflection angle of the spray cone can vary between 0 and −5 degrees for air flow rates between 0 and 35 LPM.

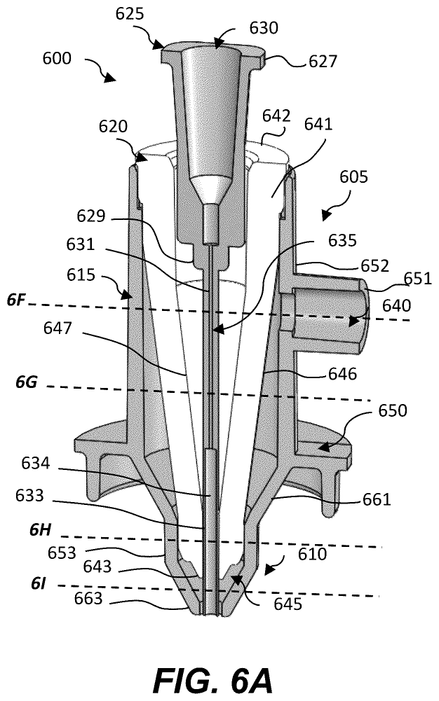

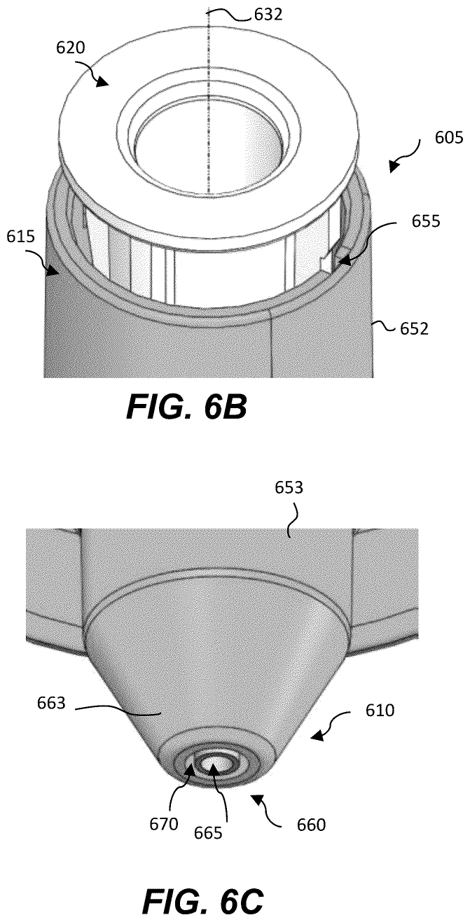

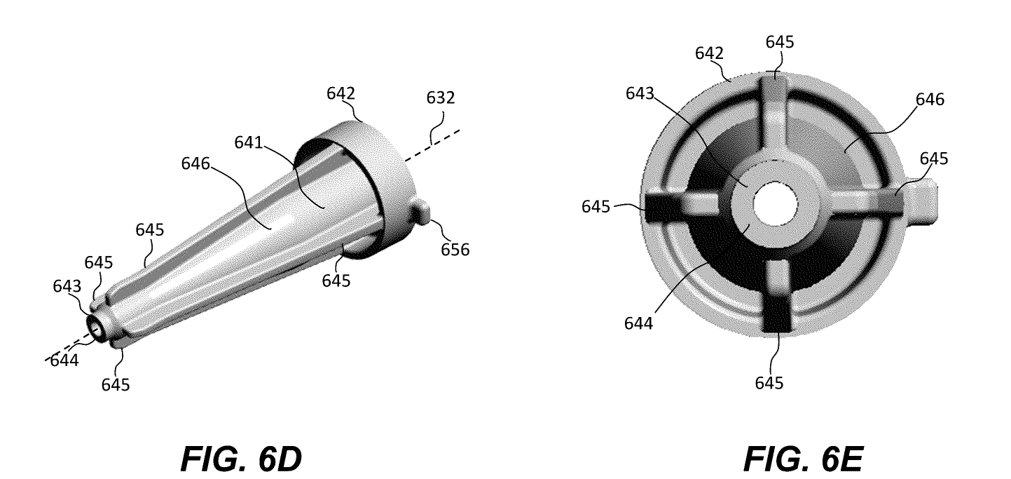

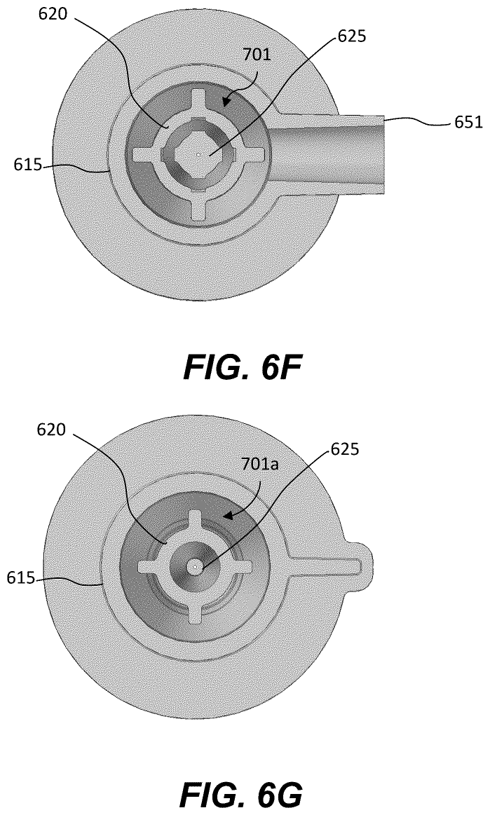

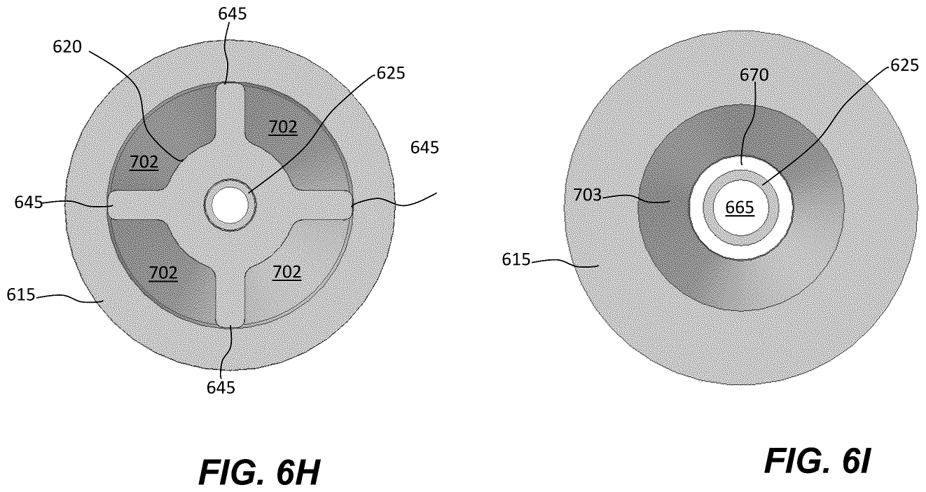

FIGS. 6A, 6B, and 6C illustrate an example implementation of the nozzle apparatus or nozzle system of FIGS. 1A-1D. The nozzle 600 includes an input end 605 and an output end 610. The nozzle 600 can include a housing 615, a sleeve 620, and a needle 625. The housing 615 can be interchangeable depending on the cells, sample fluid, cargo, or process conditions used with the nozzle 600. In this way, the nozzle 600 can be standardized during usage and other system variable can be adjusted for a specific cell, sample, cargo, or combinations thereof. A sleeve 620 can be inserted into the housing 615 and can receive the needle 625 within the sleeve 620.

As shown in FIGS. 6A-6C, the needle 625 includes a sample inlet 630, which is configured to be coupled to a sample supply (not shown). The sample inlet 630 can be fluidically coupled to a sample delivery passage 635 formed by an inner lumen of the needle 625. The needle 625 can terminate at the output end 610. The needle 625 includes a hub 627 with the sample inlet 630, which is configured for receiving a liquid sample. The hub 627 connects to a sample source via a luer connection in some embodiments. When the needle 625 is received within the housing 615, at least a portion of the hub 627 of the needle 625 is inserted through the proximal end of the housing 615. The needle 625 includes a proximal end portion 629 coupled to the hub 627 and defining a first inner diameter 631 extending along a central axis 632 (shown in FIG. 6B). The needle 625 has a distal end portion 633 connected to the proximal end portion 629 and having a sample outlet 665 for dispensing the liquid sample. The distal end portion 633 defining a second inner diameter 634 that is larger than the first inner diameter 631. The second inner diameter 634 being larger than the first inner diameter can be important in providing a highly uniform spray by advantageously mitigating siphoning effects on the sample being delivered by the needle 625. When the sample is siphoned within and down the needle lumen by lower pressure at the needle tip, caused by fast moving air, air bubbles can be introduced into the lumen of the needle and cause undesirable disruptions in the flow of the sample and the resulting atomized spray. Additionally, the first inner diameter 631 isolates the sample from the siphon effect making the backpressure applied to the sample, the primary means of controlling sample introduction and making the resulting atomized spray more controllable. In some aspects, the first diameter 631 of the needle 625 is at least 10%, 20%, 30%, 40%, 50%, 60%, 70%, 80% smaller than the second diameter 634 of the needle to provide a constriction within the needle lumen that serves to prevent undesirable siphoning effects. In some aspects, the first diameter 631 of the needle 625 has a diameter between 100 microns and 800 microns, or between 200 and 400 microns. In some aspects, the second diameter 634 has a diameter between 200 and 1500 microns, or between 500 and 950 microns.

As best shown in FIGS. 6A, 6D, 6E, the sleeve 620 includes a body 641 extending from a proximal end 642 to a distal end 643. The distal end 643 of the sleeve 620 includes a distal tip 644. The body 641 is defined by exterior walls 646 and interior walls 647. At least a portion of the interior walls 647 and the exterior walls 646 extend at an angle relative to the central axis 632 between the proximal end 642 and the distal end 643. At the proximal end 642, the interior walls 647 are dimensioned to receive and engage with or contact the hub 627 of the needle 625. At the distal end 643, the interior walls 647 are dimensioned to receive the distal end portion 633 of the needle 625.

The sleeve 620 includes a plurality of wings 645 radially spaced from one another and extending from at least a portion of the angled exterior walls 646 of the body 641. The plurality of wings 645 are equidistant from one another. In some aspects, the plurality of wings 645 includes two, three, four, five, six or more wings. The plurality of wings 645 are tapered to fit within the housing 615. In some aspects, the plurality of wings 645 have a constant width and height along the length of each of the wings 645. In some aspects, the plurality of wings 645 have a variable width and/or a variable height along the length of each of the wings 645. The plurality of wings 645 can include four wings that are spaced apart at equal 90 degree intervals around the sleeve 620.

A gap distance can be present between the plurality of wings 645 and the walls of the housing 615 in portions of the housing to help obtain desirable flow characteristics (e.g., laminar flow) of the gas (e.g., air) within the nozzle apparatus. The gap distance between the plurality of wings 645 and internal walls of the housing 615 can vary in certain locations within the housing 615. For example, the gap distance at the proximal end 642 of the sleeve may be less than the gap distance between the plurality of wings 645 and the housing 615 along a first conical portion 661 of the housing 615. The plurality of wings 645 can serve to ensure proper alignment of the sleeve 620 with the housing 615 during assembly and operation of the nozzle 600. One or more gas delivery passages can be formed between the plurality of wings 645 to subdivide the flowable gas within the cavities of the nozzle apparatus. The one or more gas delivery passages can be fluidically coupled to the gas inlet 640, as will be described in detail below in reference to a plurality of cavities.

As shown in FIGS. 6A, 6B, and 6C, the housing 615 includes multiple portions for receiving and delivering a flowable gas. The housing 615 includes a gas inlet portion 651 that defines a gas inlet 640 at the input end 605. The gas inlet 640 can be fluidically coupled to one or more gas delivery passages at the output end 610, and the gas inlet portion 651 is configured for receiving a gas at the gas inlet 640. The housing 615 includes a first cylindrical portion 652 that is fluidically coupled to the gas inlet portion 651. The first cylindrical portion 652 is configured to releasably couple with at least a portion of the sleeve 620 (e.g., the proximal end 642 of the sleeve 620). The first cylindrical portion 652 extends in a direction along the central axis 632. In some aspects, the gas inlet 640 is arranged at a right angle with respect to the first cylindrical portion 652. The gas inlet portion 651 can be arranged such that one of the wings 645 that bifurcates the flow path of the gas flowing from the gas inlet portion 651 into the other portions of the housing 615. The wing that bifurcates the flow path of the gas redirects the gas to travel around the sleeve 620 in an equal and predictable flow pattern. In some embodiments, the housing does not include a gas inlet portion, but introduces the gas in other portions of the housing 615 (e.g., the first cylindrical portion 652). In some embodiments, the gas is introduced at an acute or obtuse angle relative to the other portions of the housing (e.g., the first cylindrical portion 652).

Still referring to FIGS. 6A, 6B, and 6C, the housing 615 includes a second cylindrical portion 653. The second cylindrical portion 653 has an inner cylindrical diameter that is smaller than an inner cylindrical diameter of the first cylindrical portion 652. The second cylindrical portion 653 can be positioned distally from the first cylindrical portion 652. The second cylindrical portion 653 is configured to receive the distal end 643 of the sleeve 620. In some aspects, each of the plurality of wings 645 are tapered to fit in the housing 615 and contact at least a portion of the second cylindrical portion 653. In some embodiments, the housing includes only one cylindrical portion (e.g., first cylindrical portion 652 or second cylindrical portion 653). In some embodiments, the housing includes multiple (e.g., two, three, four, five, or more) cylindrical portions.

Referring to FIGS. 6A and 6C, the housing 615 also includes a first conical portion 661 that is fluidically coupled to and extending between the first cylindrical portion 652 and the second cylindrical portion 653. The housing 615 includes a second conical portion 663 coupled to the second cylindrical portion 653 and having the gas outlet 670 configured for dispensing the gas. The second conical portion 663 defined by a maximum inner diameter that is equal to a minimum inner diameter of the first conical portion 661. The internal walls of the first conical portion 661 and the second conical portion 663 are tapered at a same taper angle. In some aspects, the needle 625 expands to the second diameter 634 proximal to or at the first conical portion 661. In some aspects, the taper angle of the external walls 646 of the sleeve 620 are less than taper angles of the first conical portion 661 and the second conical portion 663 of the housing 615 to advantageously cause a large flow rate change in the gas flowing within the cavity defined between the sleeve 620 and housing 615. The difference in the taper angles facilitates a desired flow pattern of the gas at the gas outlet 670. Further, the difference in the taper angles facilitates an improved arrangement of the nozzle assembly 600 by maximizing the cavity volume(s) between the sleeve 620 and housing 615 and by minimizing the impact of the plurality of wings 645 on gas circulation within the cavities.

Referring to FIG. 6B, the input end 605 the housing 615 can include a keying feature 655. The keying feature 655 can be configured to mate with a corresponding keying feature 656 (see FIG. 6D) of the sleeve 620. The keying feature 655 can be configured to control and maintain rotational alignment of the sleeve 620 with respect to the housing 615. The housing 615 can also include a flanged portion 650. The flanged portion 650 can ensure that the nozzle 600 is positioned properly in a transfection device and can maintain a desired (platform independent) distance between the nozzle output end 610 and a surface containing cells to be sprayed.

FIG. 6C illustrates an isometric view of an output end of the nozzle of FIG. 6A. As shown in FIG. 6C, the output end 610 can include a sample outlet/air annulus 660. The sample outlet/air annulus 660 can include the sample outlet 665 and the gas outlet 670. The sample outlet 665 is concentrically positioned within the gas outlet 670 to ensure uniform droplet size and formation.

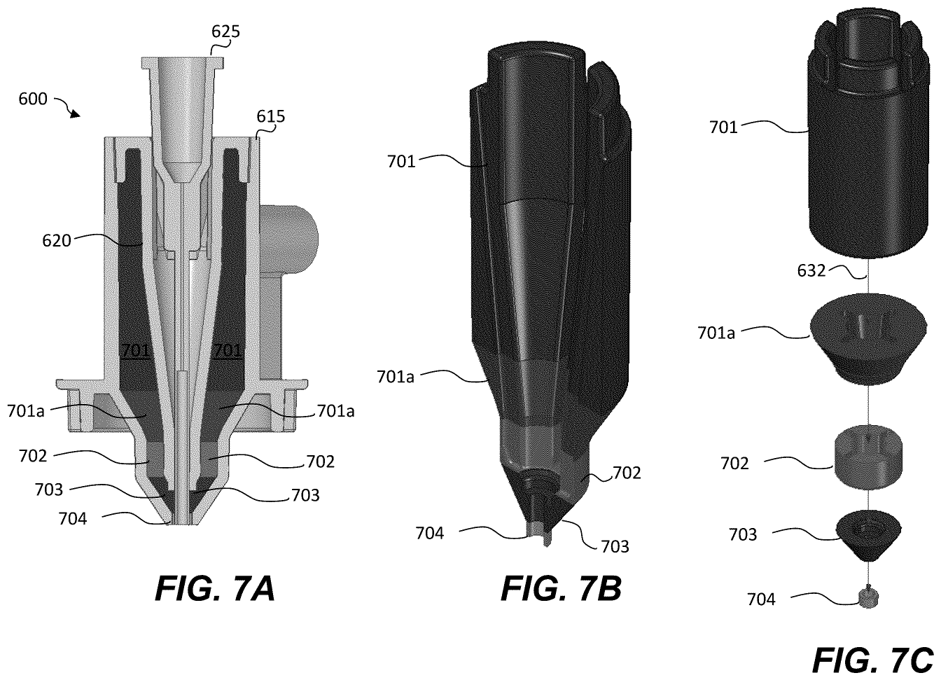

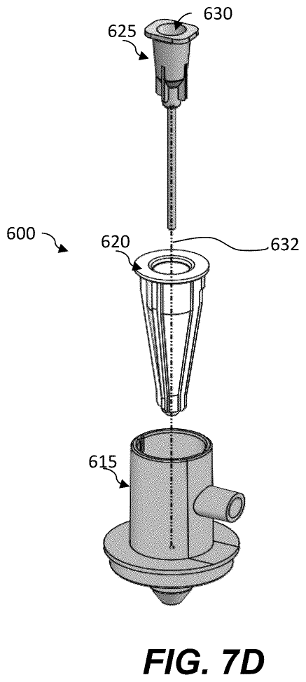

As shown in FIGS. 6F, 6G, 6H, 6I, 7A, 7B, 7C, and 7D the housing 615, the sleeve 620, and needle 625 together define cavities configured for flowing the gas. In some aspects, the cavities are contiguous with each other. The cavities can include a first cavity 701 (see FIGS. 6F, 6G, 7A-7C) that includes the gas inlet portion 651 of the housing 615 and an annular space defined by at least portion of the first cylindrical portion 652 of the housing, the first conical portion 661 of the housing 615, and the exterior walls 646 of the sleeve 620. The first cavity 701 also includes a tapered portion 701a that includes the annular space between the first conical portion 661 of the housing 615, and the exterior walls 646 of the sleeve 620. The first cavity 701 can have a volume that is between 500 mm³ and 1500 mm³, between 800 mm³ and 1300 mm³, between 900 mm³ and 1100 mm³, between 1050 mm³ and 1100 mm^{3,} and around 1080 mm³. The tapered portion 701a can have a volume that is between 50 mm³ and 250 mm³, between 100 mm³ and 200 mm³, between 130 mm³ and 190 mm³, between 170 mm³ and 180 mm³, and around 177 mm³. The first cavity 701 and the tapered portion 701a can be shaped and sized for flowing gas having either turbulent or laminar flow characteristics.

As shown in FIGS. 6H and 7A-7C, the nozzle includes a plurality of second cavities 702. The plurality of second cavities are adjacent the first cavity 701 (and tapered portion 701a). The plurality of second cavities 702 are defined by at least a portion of the second cylindrical portion 653 of the housing 615, the plurality of wings 645 and the angled exterior walls 646 of the body 641 of the sleeve 620. The plurality of second cavities 702 are coplanar to one another. The plurality of second cavities 702 are separated from each other by the plurality of wings 645 that extend into contact with the second cylindrical portion 653. In some aspects, each of the plurality of second cavities 702 define a volume that is smaller than that of the first cavity 701 (including the tapered portion 701a), the third cavity 703, or both. The plurality of second cavities 702 can have a volume that is between 20 mm³ and 80 mm³, between 30 mm³ and 70 mm³, between 40 mm³ and 60 mm³, between 50 mm³ and 60 mm^{3,} and around 55 mm³. The plurality of second cavities 702 can be shaped and sized for flowing gas having either turbulent or laminar flow characteristics. The plurality of second cavities 702 can be sized and shaped, as described above, such that the turbulent gas flow in the plurality of second cavities 702 is less turbulent than the turbulent gas flow in the first cavity 701.

As shown in FIGS. 6I and 7A-7C, the cavities include a third cavity 703. The third cavity 703 is fluidically connected to the first cavity 701 and the tapered portion 701a via the plurality of the second cavities 702. The third cavity 703 extends distally in a direction along the central axis 632 (see FIG. 7C) by a predetermined length that is distal to the distal tip 644 of the sleeve. The third cavity 703 is defined by at least a portion of the exterior walls 646 of the sleeve 620, the second conical portion 663 of the housing 615 and distal end 634 of the needle 625. The third cavity 703 can include an exit cavity 704 that surrounds the outer surface of the distal portion 633 of the needle 625. In some aspects, the exit cavity 704 and the gas outlet 670 are interchangeable. The third cavity 703 can have a volume that is larger than the volume of the second cavities 702, either collectively or individually, but smaller than the volume of the first cavity 701 (and tapered portion 701a). The third cavity 703 can have a volume that is between 5 mm³ and 50 mm³, between 10 mm³ and 45 mm³, between 20 mm³ and 40 mm³, between 25 mm³ and 35 mm³, and around 32 mm³. The third cavity 703 can be shaped and sized for flowing gas having either turbulent or laminar flow characteristics. The third cavity 703 can be sized and shaped, as described above, such that the gas flow in the third cavity 703 is laminar or at least less turbulent than the turbulent gas flow in the other cavities (e.g., first cavity 701 and/or second cavities 702).

The nozzle apparatus 600 is configured to gas propel the aqueous solution to form a spray. For example, the aqueous solution can be gas propelled from the sample outlet 665 by gas exiting the gas outlet 670. Gas propelling the aqueous solution to form a spray can be used for contacting a population of cells with the volume of aqueous solution. In some examples, the gas can include nitrogen, ambient air, or an inert gas.

FIG. 7D illustrates an exploded view of the nozzle of FIGS. 6A-6C. As shown in FIG. 7, the nozzle 600 can include the housing 615, the sleeve 620, and the needle 625. The sleeve 620 can be inserted into the housing 615. The needle 625 can be inserted into the sleeve 620.

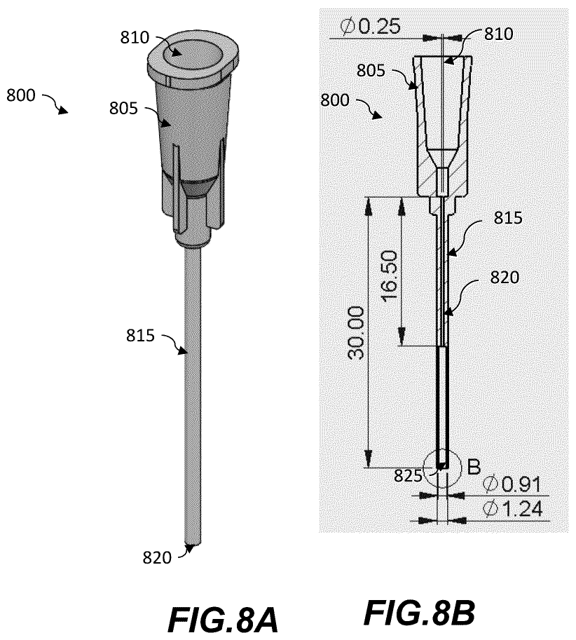

FIG. 8A illustrates an isometric view of an exemplary implementation of a needle of nozzle of FIGS. 6A-7. As shown in FIG. 8A, the needle 800 can include a body 805. The body 805 can include a sample inlet 810 through which a sample or fluid can be provided from a sample source. The sample inlet 810 can be fluidically coupled to hollow tube 815. A sample delivery passage can be formed within the hollow tube 815. The hollow tube 815 and the sample delivery passage can terminate at a sample outlet 820.

In some embodiments, the sample source can be mounted directly atop the nozzle and can be coupled to the nozzle via a check valve configured to open at a minimal operating pressure. When a pressure between 1 and 300 mbar is applied to the reservoir, sample can be fluidically conveyed to the nozzle for atomization.

FIG. 8B illustrates a cross-sectional view of the needle of FIG. 8A. As shown in FIG. 8B, hollow tube 815 can contain a sample delivery passage 820 therein. The diameter of the sample inlet 810 can be 0.2-0.3 mm, for example the diameter of the sample inlet 810 can be 0.25 mm as shown in FIG. 8B. The diameter of the sample delivery passage 820 can be 0.8-1.0 mm, for example the diameter of the sample delivery passage 820 can be 0.91 mm as shown in FIG. 8B. The diameter of the hollow tube 815 can be 1.1-1.4 mm, for example the diameter of the hollow tube 815 can be 1.24 mm as shown in FIG. 8B. The hollow tube 815 can include a small inner volume than the sample delivery passage 820. The constriction of the sample delivery passage 820 relative to the hollow tube 815 can create a high back pressure and can enable greater control for dispensing the sample and can allow for cylindrical and annular sample delivery control. As a result, the nozzle 600 can provide greater control for the duration and volume of samples dispersed in a single spray event. Additionally, the nozzle 600 can be configured with respect to a stage (e.g., a platform or membrane holding target cells) and the nozzle 600 can be aimed straight in a perpendicular manner toward the stages (and the cells thereon) without requiring the nozzle 600 to be at an angle relative to the stage.

In some embodiments, the hollow tube 815 can be 20.0-40.0 mm in length, for example the hollow tube 820 can be 30.0 mm as shown in FIG. 8B. In some embodiments, the needle 800 can include a luer connection. In some embodiments, the needle 800 can include two bonded needles. For example, as shown in FIG. 8B, a first needle can be dimensioned to be 16.50 mm and a second needle can be dimensioned to be 30.00 mm. In this way, the needle 800 can include a variable bore configured to control flow rate and prevent syphoning.

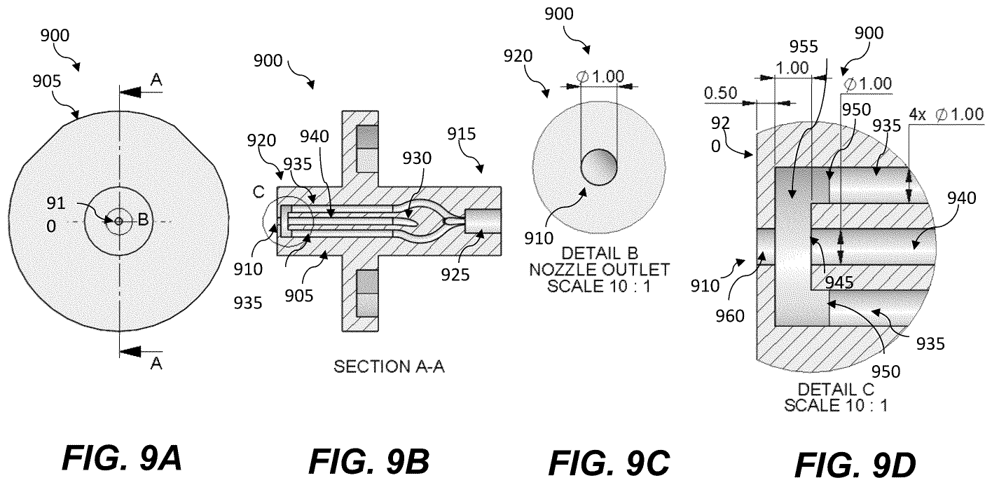

FIG. 9A illustrates a cross-sectional view of another exemplary implementation of a nozzle for use in a cell transfection system. As shown in FIG. 9A, nozzle 900 includes a housing 905 and an output orifice 910. In some embodiments, the housing 905 can be cylindrical shaped, although other shapes can be included. The output orifice 910 can be configured to dispense an atomized spray formed from a fluid and a gas that are mixed by the nozzle 900. The nozzle 900 can be configured to internally mix the sample and the gas within the nozzle 900. By controlling the high speed flow of surrounding air, the bulk of the sample can be atomized simultaneously with focusing the spray. This design can advantageously provide a symmetric and stable spray pattern, achieve smaller droplet sizes at lower flow rates, and provide spray patterns with better coverage.

FIG. 9B illustrates a cross-sectional view of the nozzle of FIG. 9A taken from the perspective of section A-A. As shown in FIG. 9B, the nozzle 900 can include an input end 915 at which a gas supply including a gas can be coupled to the nozzle 900. In some embodiments, a fluid supply including a fluid can also be coupled to the nozzle 900 at the input end 915. In some embodiments, the fluid can be a sample that is included in a sample supply coupled to the nozzle 900 at the input end 915. The nozzle 900 can also include an output end 920. The nozzle 900 can also include an output end 920 at which an atomized spray including the gas and the fluid or sample can be dispensed. The housing 905 can similarly be described to include an input end and an output end, which can respectively correspond to the input end 915 and the output end 920.

FIG. 9C illustrates a cross-sectional view of detail B of the nozzle of FIG. 9A. As shown in FIG. 9C, the output end 920 can include the output orifice 910. The nozzle 900 can be configured to mix the gas and the fluid internally within the output end 920 prior to being dispensed from the output orifice 910.

FIG. 9D illustrates a cross-sectional view of detail C of the nozzle of FIG. 9B. As shown in FIG. 9D, the gas delivery passage 935 can be coupled to the gas outlet 950. The sample delivery passage 940 can be coupled to a sample outlet 945. The sample outlet 945 and the gas outlet 950 can be coupled to a mixing chamber 955. The mixing chamber 955 can receive a sample or fluid via the sample outlet 945 and a gas via the gas outlet 950 to be mixed inside the mixing chamber 955. An output passage 960 can be coupled to the mixing chamber 955 and can provide the gas and fluid mixture as an atomized spray from the nozzle 900.

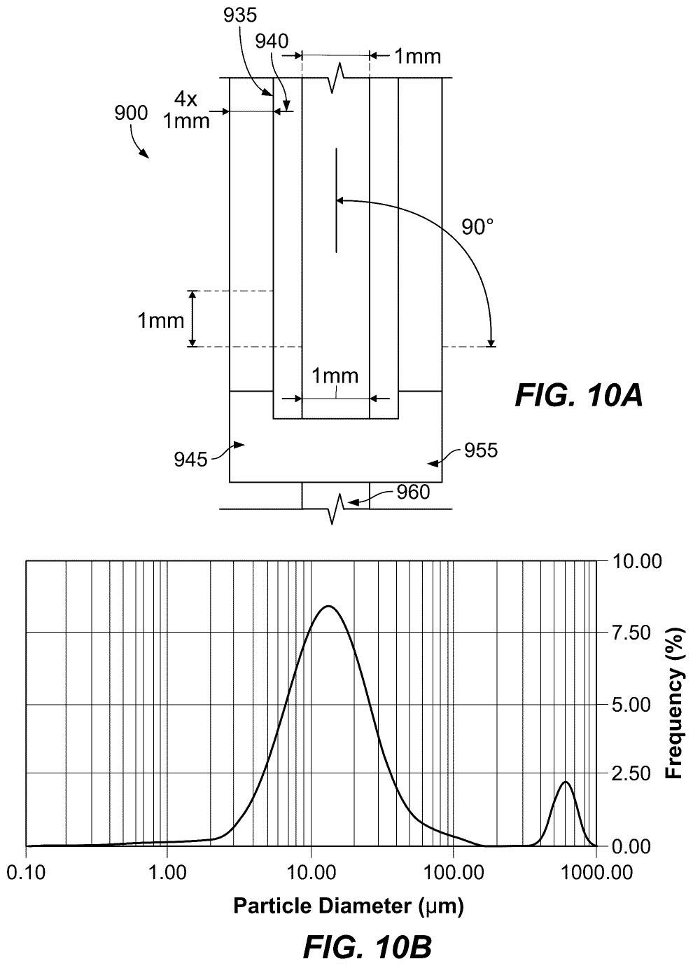

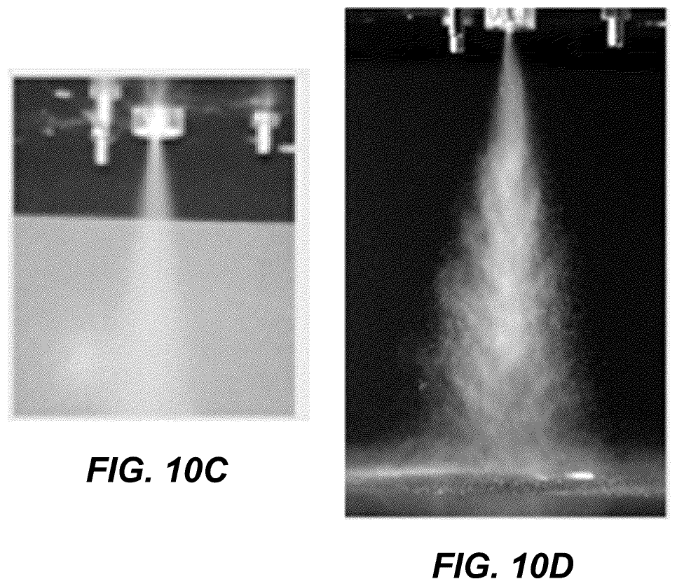

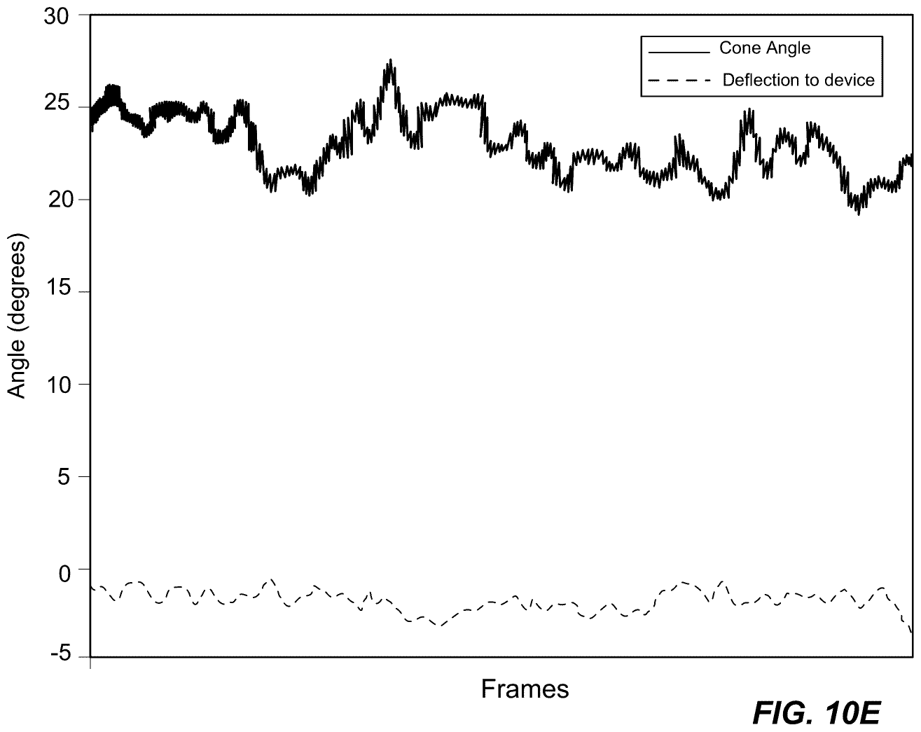

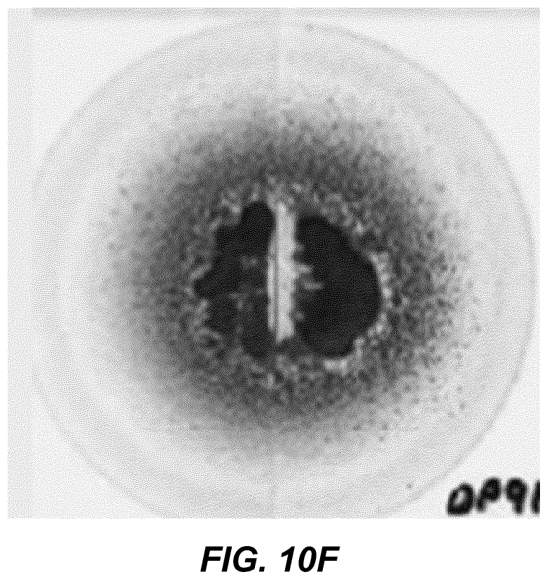

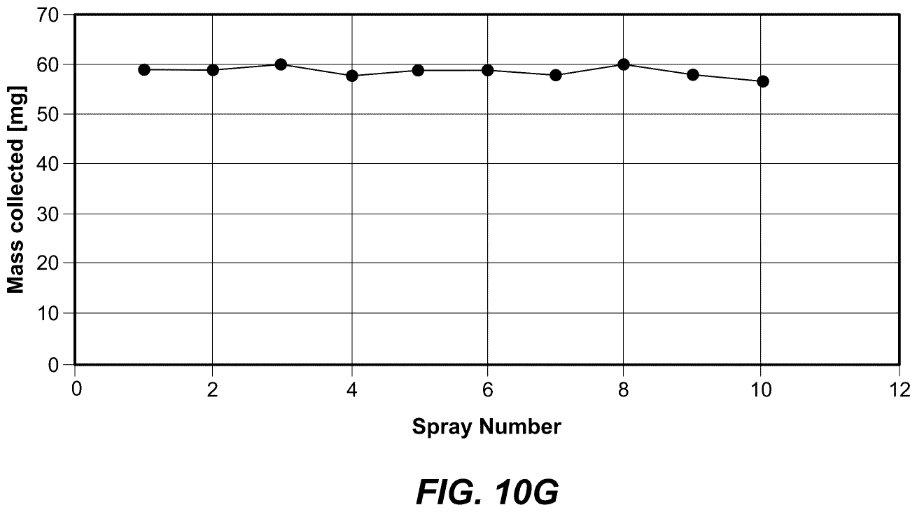

FIG. 10A illustrates a cross-sectional view of the nozzle of FIGS. 9A-9D. As shown in FIG. 10A, in some embodiments, the nozzle 900 can include a sample delivery passage 940 having a diameter of 1 mm. In some embodiments, the diameter of the sample delivery passage 940 can be the same or different than the diameter of the output passage 960. In some embodiments, the diameter of the sample delivery passage 940 (and/or the output passage 960) can be between 0.6-0.79 mm, 0.8-0.89 mm, 0.9-0.99 mm, 1.0-1.09 mm, 1.10-1.19 mm, 1.20-1.30 mm, or greater than 1.3 mm. In some implementations, the diameter of the sample delivery passage 940 can be less than 0.6 mm.

As further shown in FIG. 10A, the nozzle 900 can include a gas delivery passage 935 arranged around the sample delivery passage 940 and coupled to the mixing chamber 955. In some embodiments, the diameter of the gas delivery passage 935 can be less than 0.6 mm, 0.6-0.79 mm, 0.8-0.89 mm, 0.9-0.99 mm, 1.0-1.09 mm, 1.10-1.19 mm, 1.20-1.30 mm, or greater than 1.30 mm. In some embodiments, the diameter or size of the gas outlet 945 can be the same or different than the gas delivery passage 935.

FIG. 10B is a plot of sizes of droplets dispensed from the nozzle of FIGS. 9A-9D. As shown in FIG. 10B, the size of the droplets dispensed from the nozzle 900 can be about 10-12 μm. In some embodiments, the size of the droplets can be between 0-9.9 μm, 10-10.9 μm, 11-11.9 μm, 12-12.9 μm, 13-13.9 μm, 14-14.9 μm, 15-15.9 μm, 16-16.9 μm, or 17-18 μm. The example nozzle can advantageously create uniformly sized droplets, which can more effectively provide uniform distribution of a sample to cells. The droplet size achieved using the example nozzle has been shown to result in good transfection of cells compared to some existing nozzle designs. In some embodiments, 80% or more of the sample droplets produced by the output orifice 910 can have droplet sizes in the ranges described above.

FIG. 10C is an image of a spray dispensed from the nozzle of FIGS. 9A-9D. As shown in FIG. 10C, the spray is atomized and well developed to form a symmetrical pattern that can result in uniform distribution of the atomized cargo or sample across the target surface of cells.

FIG. 10D is another image of the spray dispensed from the nozzle of FIGS. 9A-9D. As shown in FIG. 10D, the cone of spray emitted from nozzle 900 is perpendicular to the surface below the nozzle 900 and is minimally deflected relative to a central axis of the nozzle 900. In this way, the cone of spray can consistently cover a surface on which cells are located for permeabilization without requiring adjustment or additional equipment to correct for deflection angles of the cone of spray.

FIG. 10E is a plot of core spray angle and deflection to device axis of the spray dispenses from the nozzle of FIGS. 9A-9D. As shown in FIG. 10E, the cone angle (upper plot line) can vary between 20 and 30 degrees. The cone angle can be measured from the center axis of the cone to an outer edge of the cone measured at the surface that the cone of spray is contacting. In some embodiments, the cone angle can be between 20-21.9 degrees, 22-23.9 degrees, 24-25.9 degrees, 26-27.9 degrees, or 28-30 degrees. The angle of the deflection relative to the nozzle 100 (lower plot line) can vary between 0 and −5 degrees.

FIG. 10F is an image of a spray pattern dispensed from the nozzle of FIGS. 9A-9D. As shown in FIG. 10F, the spray pattern emitted by nozzle 900 can be uniform distribution with concentrations of droplets that are concentrically arranged.

FIG. 10G is a plot of a mass of material in the spray dispensed from the nozzle of FIGS. 9A-9D. As shown in FIG. 10G, the mass (or dosage) of material provided in the atomized spray of nozzle 100 can vary between 55 and 66 mg. In some embodiments, the mass or dosage can be between 40-44.9 mg, 45-49.9 mg, 50-54.9 mg, 55-59.9 mg, 60-64.9, or 65-70 mg. The material or sample can be provided onto the cells with an impact pressure between 15-19.9 Pa, 20-24.9 Pa, 25-29.9 Pa, 30-34.9 Pa, 35-39.9 Pa, 40-44.9 Pa, 45-49.9 Pa, 50-54.9 Pa, or 55-59.9 Pa. Exercising precise control over the mass of sample or material delivered to cells is an important objective for dose control in cell therapy manufacture or cell transfection systems. Appropriate dose control can be important to ensure that transfection can occur at the optimum level, but also to reduce the potential for cell damage due to over exposure to a cargo or sample. Similarly, controlling the impact pressure can protect cells from excessive damage while providing enough pressure to facilitate membrane perturbation.

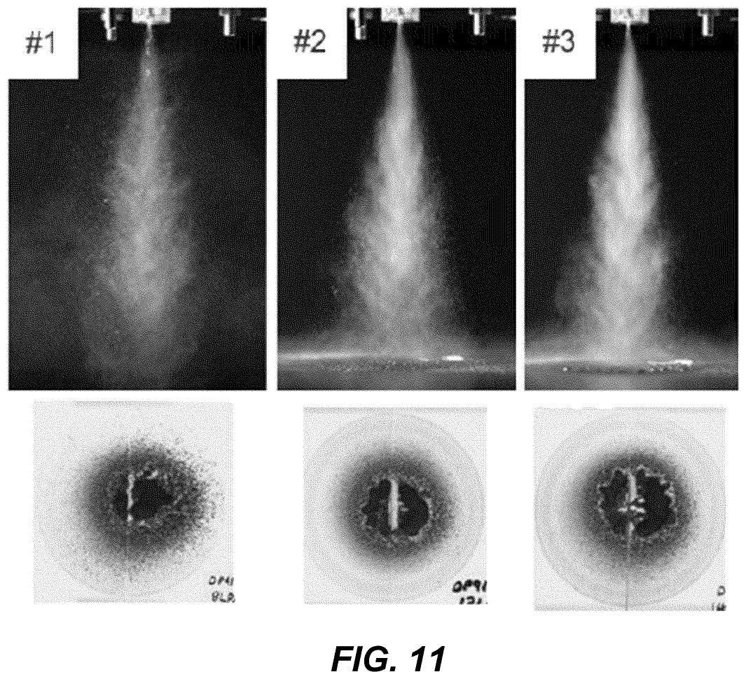

FIG. 11 includes images of spray patterns dispensed at various flow rates from the nozzle of FIGS. 9A-9D. As shown in FIG. 11, the nozzle 900 can emit different shaped sprays at different flow rates. For example, the settings in table 2 correspond to cone images 1-3 and the related spray pattern images shown below each cone image.

TABLE 2

| | | | |
|---|---|---|---|
| Setting | #1 | #2 | #3 |

| | | | | | | |
|---|---|---|---|---|---|---|
| Air Pressure | 540 | mbar | 1170 | mbar | 1425 | mbar |
| Air flow rate | 8 | LPM | 13 | LPM | 14.7 | LPM |
| Impact Pressure | 9.1 | Pa | 21.2 | Pa | 27.3 | Pa |

As shown in FIG. 11, the appearance of the spray cone changes as air pressure changes. The structure of the nozzle can allow the spray to be tuned so that the balance between impact pressure and droplet distribution can be optimized for the cell type being targeted.

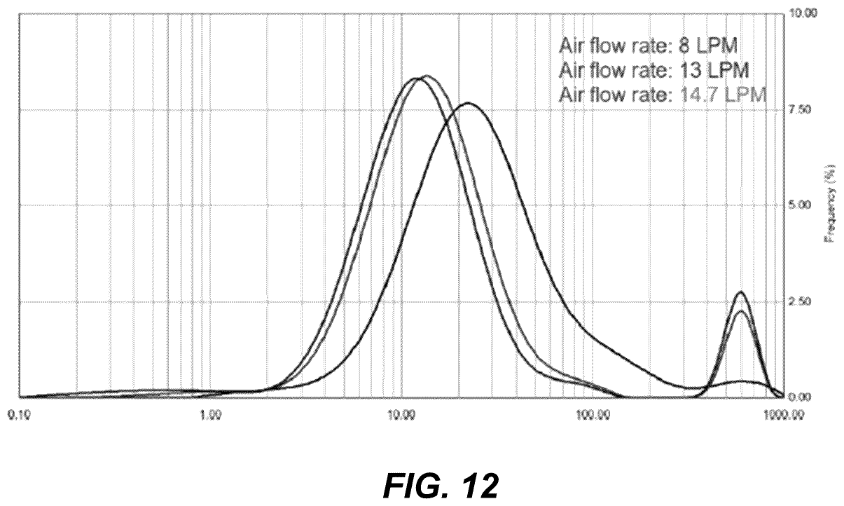

FIG. 12 is a plot of sizes of droplets dispensed from the nozzle of FIGS. 9A-9D at various flow rates. As shown in FIG. 12, the nozzle 900 can dispense droplets that have different sizes when the fluid or spray is dispensed at different flow rates. For example, as shown in FIG. 12, at lower flow rates the droplet size is larger. At faster flow rates, the droplet size is smaller.

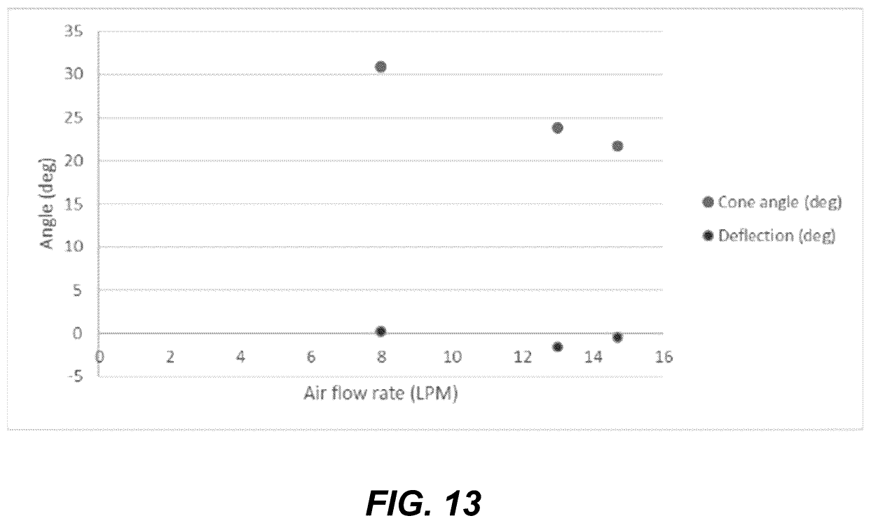

FIG. 13 is a plot of cone angle and deflection angle of a spray dispensed from the nozzle of FIGS. 9A-9D at various air flow rates. As shown in FIG. 13, the cone angle of the spray emitted from nozzle 900 can vary between 20 and 35 degrees for air flow rates between 8 and 16 LPM. The deflection angle of the spray cone can vary between 0 and −5 degrees for air flow rates between 8 and 16 LPM.

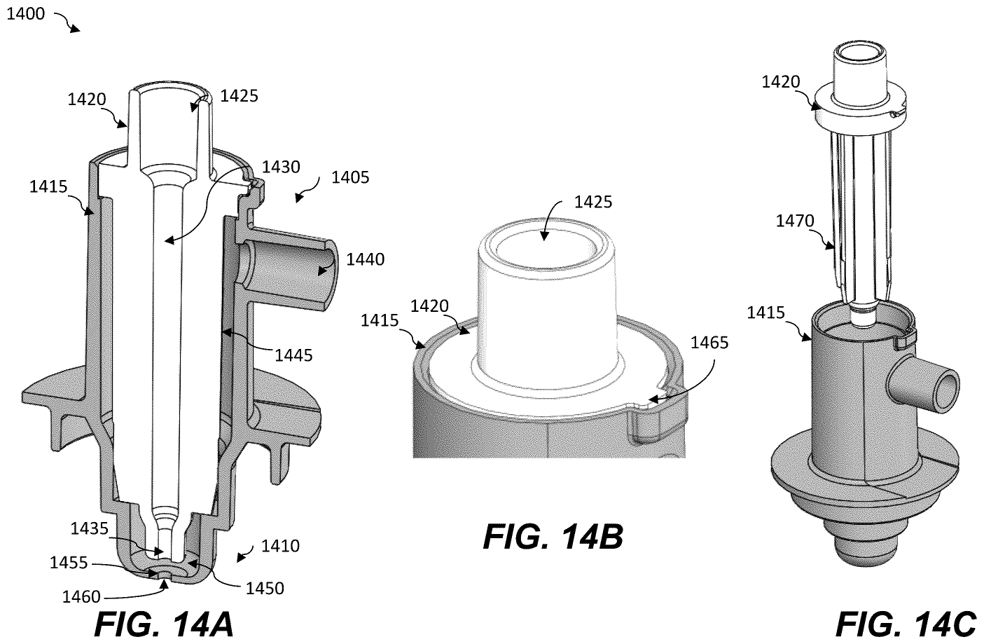

FIG. 14A illustrates an isometric cross-sectional view of an exemplary implementation of the nozzle of FIGS. 9A-9D. As shown in FIG. 14A, the nozzle 1400 can include an input end 1405 and an output end 1410. The nozzle 1400 can include a housing 1415. A sleeve 1420 can be arranged within the housing 1415 and can include a sample inlet 1425. The sample inlet 1425 can couple to a sample supply. The sample inlet 1425 can be fluidically coupled to a sample delivery passage 1430 formed within the sleeve 1420. The sample delivery passage 1430 can terminate at a sample outlet 1435.

As further shown in FIG. 14A, the nozzle 1400 can include a gas inlet 1440 formed in the housing 1415 at the input end 1405. The gas inlet can be fluidically coupled to a gas delivery passage 1445. The gas delivery passage 1445 can be fluidically coupled to a mixing chamber 1450. The mixing chamber 1450 can be further fluidically coupled to the sample outlet 1435. An output passage 1455 can provide an atomized spray of gas and fluid (or sample) that has been mixed in the mixing chamber 1455. The output passage 1455 can be included in the output orifice 1460 from which the atomized spray is dispensed from the nozzle 900.

FIG. 14B illustrates an isometric view of an input end of the nozzle of FIG. 14A. As shown in FIG. 14B, at the input end 145 the housing 1415 can include an alignment mechanism 1465 configured to ensure that fin members located on an external surface of the sleeve 1420 are positioned with a particular orientation relative to the housing 1415.

FIG. 14C illustrates an exploded view of the nozzle of FIG. 14A. As shown in FIG. 14C, the sleeve 1420 can include one or more fin members 1470. The fin members 1470 can be formed on the exterior surface of the sleeve 1420 so as to provide one or more gas delivery passages 1445 between the exterior surface of the sleeve 1420 and the interior surface of the housing 1415 (as shown in FIG. 14A). The fin members 1470 can be configured to control the depth and concentricity of the sleeve 1420 within the housing 1415. As shown in FIG. 14C, the sleeve 1420 can be inserted into the housing 1415 to form the nozzle 1400 as shown in its assembled state in FIG. 14A.

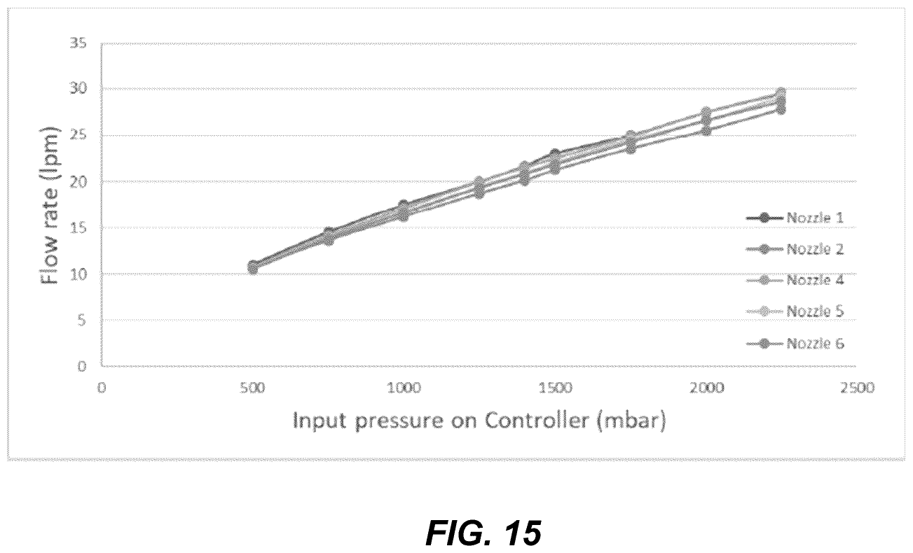

FIG. 15 is a plot of flow rates at various input pressures and operating points by the nozzle of FIGS. 1A-1D. The operating points are summarized in table 3.

TABLE 3

| | | Input | Mass/spray | Dv(50) | Impact | |
|---|---|---|---|---|---|---|
| Nozzle # | Input air | sample | (mg) | (um) | pressure | |
| 100-1 | 1800 mbar | 130 mbar | 56.5 | 14.49 um | 41 | Pa |
| | | | (average | | | |
| | | | over 5 | | | |
| | | | sprays) | | | |
| 100-2 | 1900 mbar | 120 mbar | 50.3 | 15.92 um | 43.6 | Pa |
| | | | (average | | | |
| | | | over 5 | | | |
| | | | sprays) | | | |
| 100-4 | 1740 mbar | 100 mbar | 55.3 | 16.71 um | 39.5 | Pa |
| | | | (average | | | |
| | | | over 5 | | | |
| | | | sprays) | | | |
| 100-5 | 1800 mbar | 130 mbar | 52.3 | 14.79 um | 41.1 | Pa |
| | | | (average | | | |
| | | | over 5 | | | |
| | | | sprays) | | | |
| 100-6 | 1775 mbar | 90 mbar | 54.2 | 15.26 um | 40.2 | Pa |
| | | | (average | | | |
| | | | over 5 | | | |
| | | | sprays) | | | |

In the plot shown in FIG. 15, the change in flow rate is relatively consistent at increasing input pressures for a variety of embodiments of nozzle 100. The plot illustrates the relationship between pressure and flow rate for nozzle 100 and how droplet size can be modified by increasing air flow rates in a predictable manner.

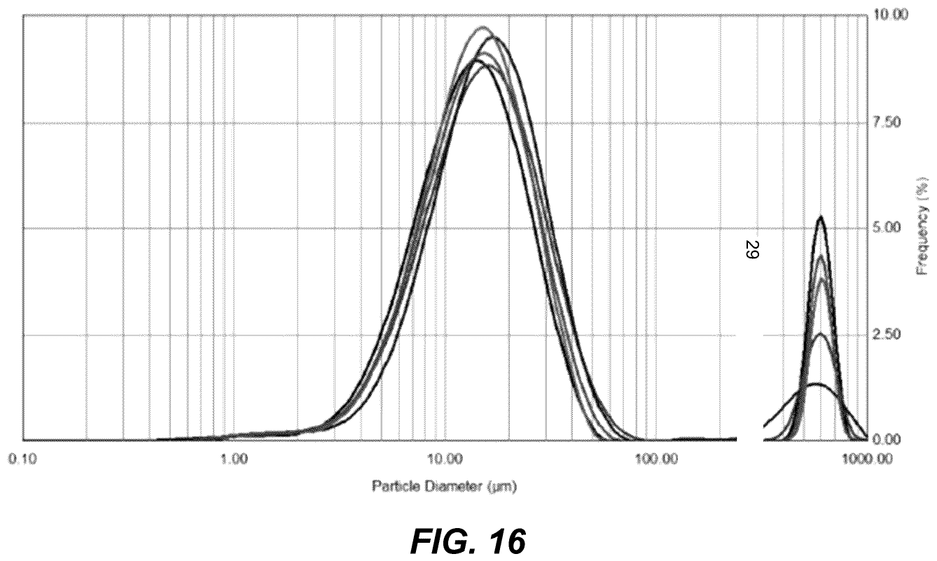

FIG. 16 is a plot of sizes of droplets dispensed from exemplary implementations of the nozzle of FIGS. 1A-1D. The plot lines correspond to the operating conditions and the various embodiments of nozzle 100 shown and described in table 3. The plot illustrates how nozzle 100 can enable drop size control by tuning the input pressure. As a result, nozzle 100 can advantageously be used to alter droplet size by manipulating input parameters, such as flow rate and/or pressure.

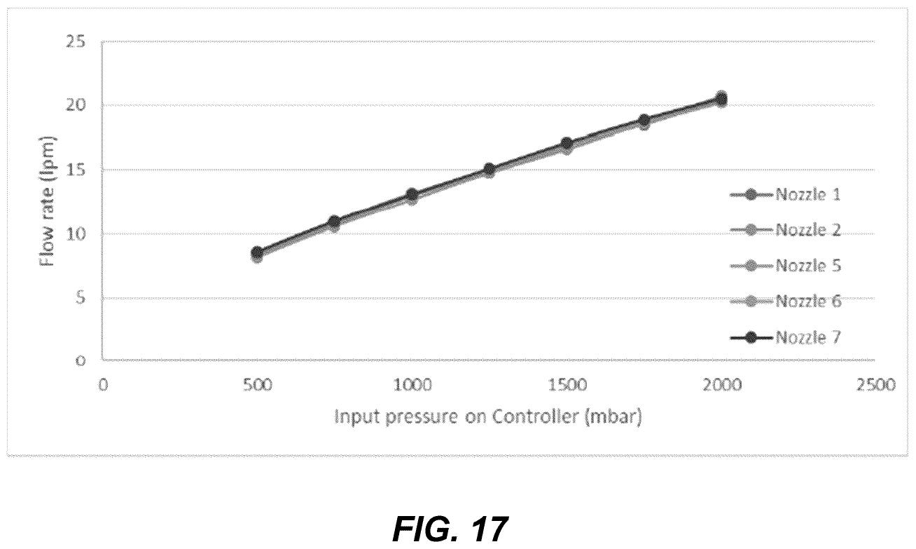

FIG. 17 is a plot of flow rates at various input pressures and operating points by the nozzle of FIGS. 9A-9D. The operating points are summarized in table 4.

TABLE 4

| | | Input | Mass/spray | Dv(50) | | Impact |
|---|---|---|---|---|---|---|
| Nozzle # | Input air | sample | (mg) | (um) | | pressure |
| 900-1 | 1500 mbar | 925 mbar | 52.4, 54.8, | 12.68 | um | 33.6 Pa |
| | | | 56.4 | | | |
| | | | (1 spray, 3 | | | |
| | | | consecutive | | | |
| | | | measurements) | | | |
| 900-2 | 1500 mbar | 925 mbar | 52.8, 51.4, | 11.9 | um | 33.5 Pa |
| | | | 51.8 | | | |
| | | | 1 spray, 3 | | | |
| | | | consecutive | | | |
| | | | measurements) | | | |
| 900-5 | 1550 mbar | 965 mbar | 52.6, 52.3, | 11.6 | um | 34.4 Pa |
| | | | 52.5 | | | |
| | | | (1 spray, 3 | | | |
| | | | consecutive | | | |
| | | | measurements) | | | |
| 900-6 | 1500 mbar | 965 mbar | 52.5, 52.1, | 13.47 | um | 33.2 Pa |
| | | | 52.4 | | | |
| | | | (1 spray, 3 | | | |
| | | | consecutive | | | |
| | | | measurements) | | | |
| 900-7 | 1500 mbar | 965 mbar | 54.8, 55.9, | 12.2 | um | 34.1 Pa |
| | | | 52.6 | | | |
| | | | (1 spray, 3 | | | |
| | | | consecutive | | | |
| | | | measurements) | | | |

The plot shown in FIG. 17 illustrates the change in flow rate is relatively consistent at increasing input pressures for a variety of embodiments of nozzle 900. The plot illustrates the relationship between pressure and flow rate for nozzle 900 and how droplet size can be modified by increasing air flow rates in a predictable manner.

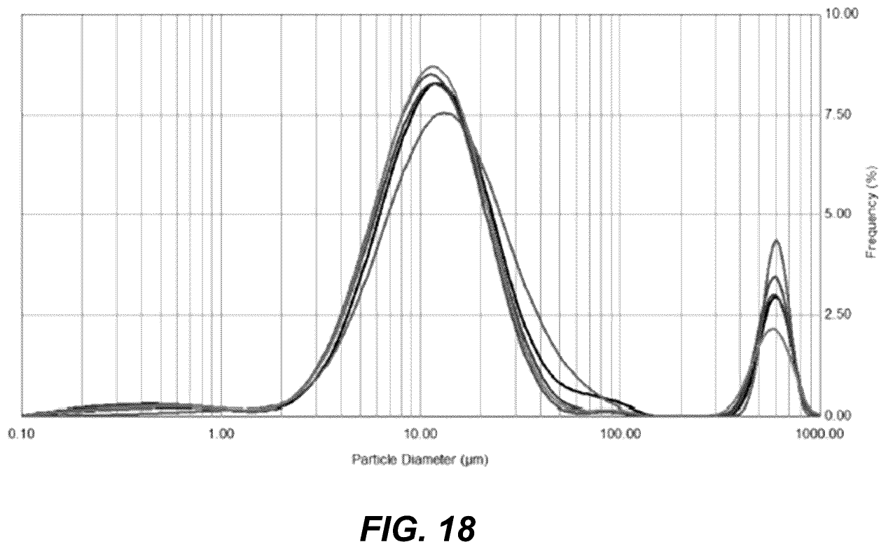

FIG. 18 is a plot of sizes of droplets dispensed from exemplary implementations of the nozzle of FIGS. 9A-9D. The plot lines correspond to the operating conditions and the various embodiments of nozzle 900 shown and described in table 4.

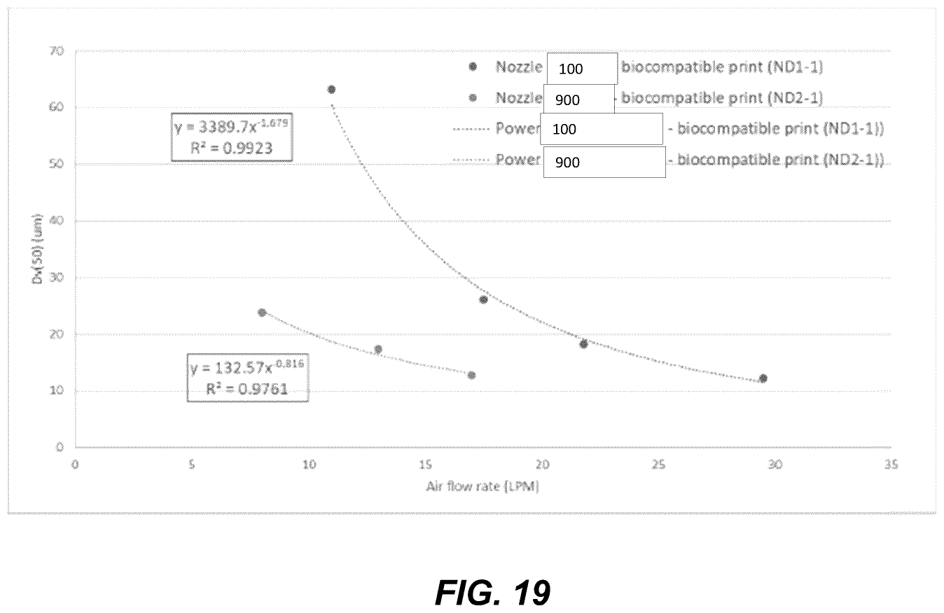

FIG. 19 is an interpolation plot illustrating relationships between sizes of droplets dispensed from the nozzles of FIGS. 1A-1D and 9A-9D for various air flow rates and manufacturing materials. As shown in FIG. 19, the particulate sizes for provided by nozzle 100 and 900 are shown as a function of air flow rate in LPM. Nozzle 100 achieves higher particulate size at lower air flow rates compared to nozzle 900. As air flow rates increase, particulate size is reduced for both nozzles.

As shown in the plot of FIG. 19, two different operational spaces exist for the designs of nozzle 100 and nozzle 900 with respect to their relationship between droplet size and airflow rate. The plot illustrates that each nozzle can achieve different droplet sizes and impact pressures, which can allow tuning of the transfection system to the needs of the cell type of interest. The power plot lines illustrate a regression fit for the relationship between the droplet size and the air flowrate for each of the nozzles 100 and 900. Understanding and modelling this relationship using a regression fit can allow a user to predict what droplet size can result from the use of a given air flowrate for each of nozzles 100 and 900.

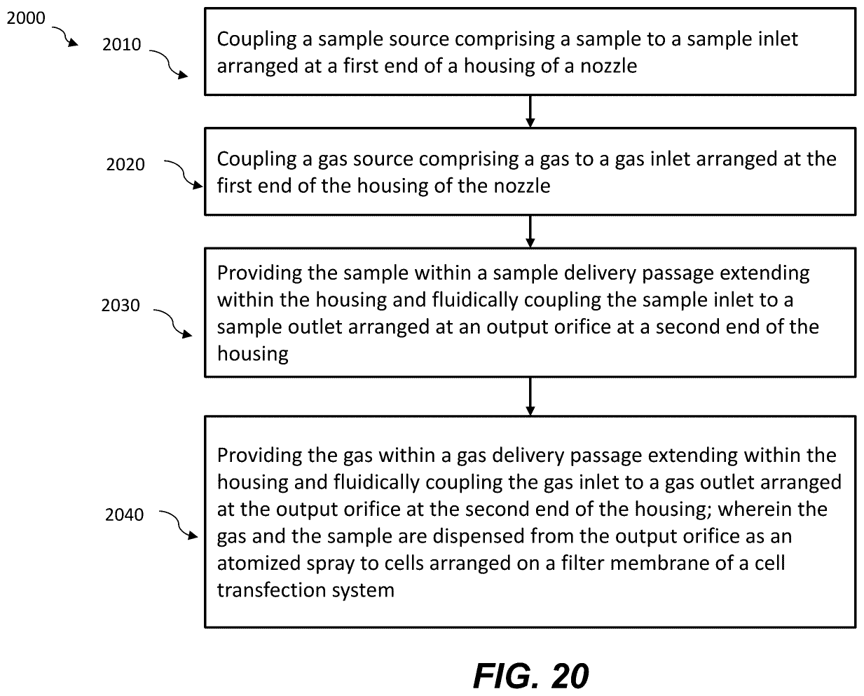

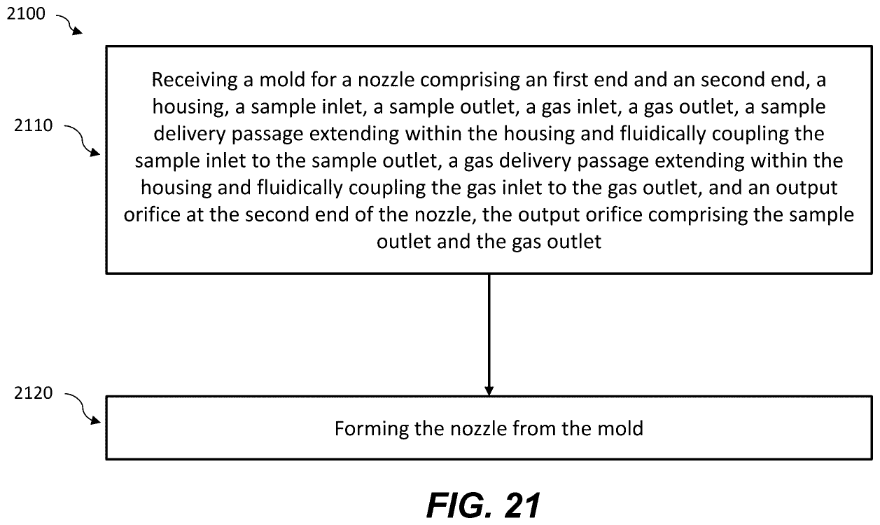

FIG. 20 is a process flow diagram illustrating an exemplary process for using the nozzle of FIGS. 1A-1D or 9A-9D in a cell transfection system. The process 2000 can be described in relation to embodiments of the nozzles 100 and 600 shown and described in relation to FIGS. 1A-1D and 6A-8B, as well as nozzles 900 and 1400 shown and described in relation to FIGS. 9A-9D and 14A-14C. As shown in FIG. 20, at 2010 the process 2000 can include coupling a sample source including a sample to a sample inlet arranged at a first end of a housing of a nozzle. The first end can be the input end of the nozzle or the housing. The sample source can include a sample or a fluid containing a sample that is to mixed with a gas using the nozzle to form an atomization spray. In some embodiments, the sample can include ethanol. In some embodiments, the sample can include a suspension of bimolecular cargo, such as mRNA, DNA, gene editing tools, or the like. In some embodiments, the sample can include a biological buffer, phosphate buffered saline (PBS), or water for injection (WFI). A variety of samples or fluids and sample or fluid supplies can be used to form an atomized spray using embodiments of the nozzle described herein.

At 2020, the process 2000 can include coupling a gas source including a gas to a gas inlet arranges at an input end of the housing of the nozzle. The gas source can include a gas to be mixed with the sample or fluid received within the nozzle from the sample source. The gas can provide a medium in which the sample or fluid can be mixed using the nozzle to form the atomization spray. In some embodiments, the gas can include air. In some embodiments, the gas can include nitrogen. A variety of gases and gas supplies can be used to form an atomized spray using embodiments of the nozzle described herein.

At 2030, the sample can be provided within a sample delivery passage that can extend within the housing. The sample delivery passage can fluidically couple the sample inlet to a sample outlet that can be arranged at an output orifice at a second end of the housing. The second end can be the output end of nozzle or the housing. The sample can be provided via the sample delivery passage to the sample outlet to be internally or externally mixed with the gas at the output end of the nozzle described herein.

At 2040, the gas can be provided within a gas delivery passage that can extend within the housing. The gas delivery passage can fluidically couple the gas inlet to a gas outlet that can be arranged at an output orifice at the output end of the housing of the nozzle. The gas and the sample can be dispensed from the output orifice as an atomized spray. In some embodiments, the atomized spray can be provided to cells arranged on a filter plate or a filter membrane of a cell transfection system or a reverse permeabilization platform described herein.

The sample and the gas can be provided to the nozzle at various input pressures such that the atomized spray dispensed from the nozzle described herein can be released from the nozzle with a desired output pressure. The input pressures of the sample and the gas can be controlled via one or more pumps and one or more controllers configured within the cell transfection system or the reverse permeabilization platform described herein. For example, the sample can be provided at a pressure between 100-119 mbar, 120-129 mbar, 130-139 mbar, 140-149 mbar, 150-199 mbar, 200-249 mbar, 250-349 mbar, 350-399 mbar, 400-449 mbar, 450-499 mbar, 500-549 mbar, 550-599 mbar, 600-649 mbar, 650-699 mbar, 700-750 mbar, 751-800 mbar, 801-899 mbar, or 900-1000 mbar. In some embodiments, the pressure can be greater than 100 mbar or less than 100 mbar. Other pressures can be provided. In some implementations, the gas can be provided at a pressure. In some embodiments, the gas can include sterile filtered air. In some embodiments, the nozzles described herein can be configured for single use or multiple uses.

FIG. 21 is a process flow diagram illustrating an exemplary process for manufacturing the nozzle of FIGS. 1A-1D or 9A-9D in cell transfection system. The process 2100 will be described in relation to embodiments of the nozzles 100 and 600 shown and described in relation to FIGS. 1A-1D and 6A-8B, however the process 2100 also applies to the manufacture of nozzle 900 shown and described in relation to FIGS. 9A-9D and 14A-14C.

As shown in FIG. 21, at 2110 the process 2100 can include receiving a mold for a nozzle including an first end and a second end. The first end can correspond to an input end of the nozzle or the housing. The second end can correspond to an output end of the nozzle or the housing. The nozzle can also include a housing, a sample inlet, a sample outlet, a gas inlet, a gas outlet, a sample delivery passage extending within the housing. The sample delivery passage can fluidically couple the sample inlet to the sample outlet. The nozzle can also include a gas delivery passage extending within the housing. The gas delivery passage can fluidically couple the gas inlet to the gas outlet. The nozzle can also include an output orifice including the sample outlet and the gas outlet.

At 2120, the process 2100 can include forming the nozzle from the mold. The mold can be used in an injection molding process or manufacturing system to form the nozzle. The nozzle can be formed from a polymer material, such as a photopolymer material. In some embodiments, the nozzle can be formed from a biocompatible material. The biocompatibility of the material can be defined according to ISO 10993 and can correspond to an ability of a material or a device to perform with an appropriate host response in a specific situation. The biocompatibility of a material can depend on the specific application for which it will be used. In this regard, a material could be biocompatible for one application but not for another.

Typical biocompatible materials can include biocompatible plastics such as medical grades of polyvinyl chloride, polyethylene, polycarbonate, polyether ether ketone, polyetherimide, polypropylene, polysulfone, and polyurethane. In some embodiments, the nozzle described herein can be formed via additive manufacturing, tube welding, or tube bonding.

The polymeric material used to form the nozzle described herein can avoid wetting of internal surfaces and can preserve the amount of sample used. The nozzle can avoid “wall effects” where sample can be lost or adhered within internal portions of the nozzle.

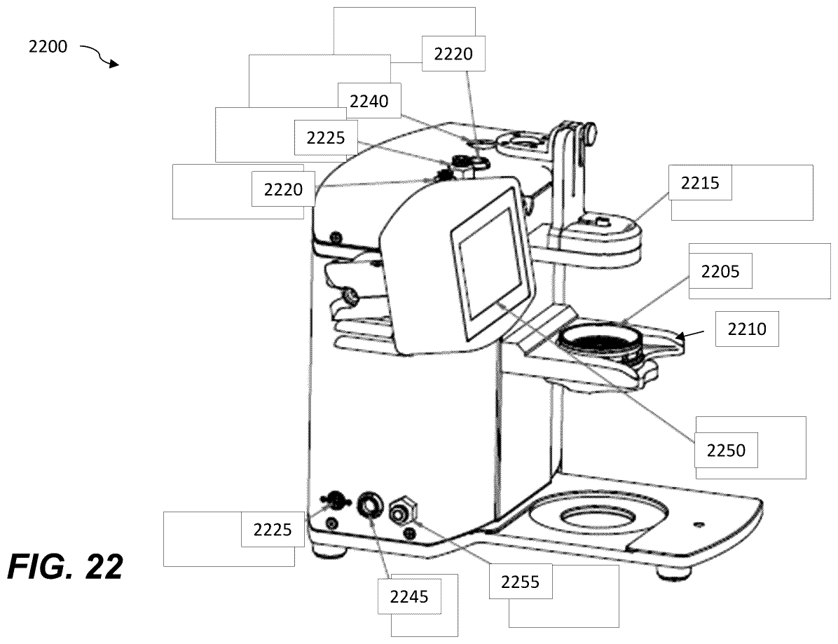

FIG. 22 is an isometric view of a computer aided design (CAD) drawing illustrating an example embodiment of a cell transfection system (CTS) 2200 according to some embodiments disclosed herein. In some embodiments, the CTS 2200 can be a reversible permeabilization platform. In some embodiments, the nozzles shown and described in relation to FIGS. 1A-1D and 9A-9D can be configured for use in the CTS 2200.

The CTS 2200 includes a pod 2205 configured to be received and positioned within a pod nest 2210. The pod 2205 can provide a processing surface, via a filter plate, on which cells can be provided for treatment and processing. For example, the filter plate can be configured to receive a filter for use in forming a monolayer of cells to be processed using the CTS 2200. In some embodiments, the filter plate can include a filter membrane.

The pod 2205 can be received and positioned within the pod nest 2210. In some embodiments, the nozzle nest 2215 can be a fixed distance above the pod 2205. The nozzle nest 2215 can be a fixed distance from the pod nest 2210 to reduce the number of variables or degrees of freedom available to the user thereby providing a system that is easier to use. For example, the nozzle nest 2215 can be fixed so that a nozzle or nozzle held in the nozzle nest 2215 can be positioned 65 mm to 80 mm above the pod 2205. In some implementations, the nozzle nest 2215 can be positioned so that a nozzle or nozzle held in the nozzle nest 2215 is positioned about 75 mm above the pod 2205. The pod nest 2210 can include a circular opening to receive the pod 2205. A lower portion of the pod 2205 can be mated to the filter plate by coupling the lower portion with a portion of the filter plate extending through the circular opening of the pod nest 2210. The pod nest 2210 can provide support to the pod 2205 and can maintain the position of the pod 2205 during cell processing using the CTS 2200. For example, the pod nest 2210 can maintain the position of the pod 2205 to ensure the treatment surface of the pod 2205, e.g., the filter plate, is sufficiently located to receive adequate amounts of permeabilizing solution.

As further shown in FIG. 22, the CTS 2200 includes a nozzle nest 2215. The nozzle nest 2215 can include a nozzle coupled to a permeabilizing solution source configured within the CTS 2200. The nozzle can atomize the permeabilizing solution to provide the permeabilizing solution to the pod 2205 (e.g., in the form of a spray) to process or treat cells configured on the filter plate of the pod 2205. The nozzle nest 2215 can be coupled to the permeabilizing solution source via a valve connector 2220, such as a clippard value connector. The nozzle configured within the nozzle nest 2215 can be configured to provide the permeabilizing solution to the pod 2205 at a predetermined pressure. The CTS 2200 also includes a sample pressure connector 2225 and an air pressure connector 2230. The valve connector 2220 serves to control delivery solution application to nozzle. The sample pressure connector 2225 pressurizes the gas above the fluid in the Eppendorff reservoir to drive the sample into the nozzle. The gas pressure connector 2230 supplies pressurized gas to the nozzle.

The CTS 2200 also includes a power input 2235. In some embodiments, the power input 2235 can include a 2 channel direct current (DC) 24V power input 2235. The power input 2235 can be electrically coupled to the On/Off switch 2240. The CTS 2200 also includes a human machine interface (HMI) cable coupling 2245. The HMI cable coupling 2245 can be electrically coupled to the HMI 2250. The HMI 2250 can include a display, at least one data processor, and input devices configured to control operation of the CTS 2200 and to perform the methods of cell treatment via reversible permeabilization described herein. In some embodiments, the HMI 2250 can include a touch screen interface. In some embodiments, the HMI 2250 can include process guides, laboratory timers, and the like. The HMI cable coupling 2245 can be configured to couple the HMI 2250 to a computing device that is located separately from the CTS 2200. In this way, data can be imported to or exported from the CTS 2200.

The CTS 2200 further includes an air supply coupling 2255. The air supply coupling 2255 can couple the CTS 2200 to an air supply. The air supply can be used to provide air, via the air supply coupling 2255, for use in configuring an amount of air to be provided with the permeabilizing solution to the pod 2205.

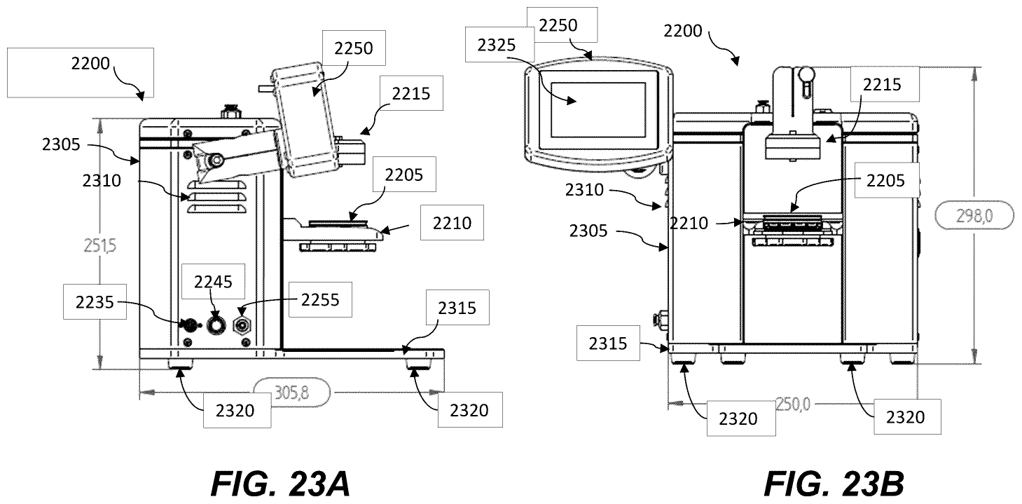

FIG. 23A is a side view of the CTS 2200 shown in FIG. 22. As shown in FIG. 23A, the CTS 2200 can include an enclosure 2305. The enclosure 2305 can include a number of cutouts corresponding to the power input 2235, the HMI cable coupling 2245, and the air supply coupling 2255. Additional cutouts can be provided within the enclosure 2305 without limitation. For example, the enclosure 2305 can include a plurality of vents 2310. The enclosure 2305 can be affixed to a base plate 2315. The base plate 2315 can include a plurality of feet 2320. In some embodiments, the feet 2320 can be plastic and can include friction-reducing materials to secure the CTS 2200 on a surface.

FIG. 23B is a front view of the CTS 2200 shown in FIG. 22. As shown in FIG. 23B, the CTS 2200 can include an HMI 2250 and the HMI 2250 can include a display 2325. The display 2325 can provide visualizations of data and user-interface controls corresponding to one or more aspects of operation of the CTS 2200. For example, in some embodiments, the display 2325 can provide touch screen controls configured to perform one or more operations of methods of reversibly permeabilizing cells. In some embodiments, the HMI 2250 can include a timer and the timer, as well as timer controls, can be displayed via the display 2325.

In some implementations, the CTS 2200 can include a spray-guard device to contain atomization (e.g., overspray). In one example, the spray-guard is transparent, demi-cylindrical device that has the same internal diameter as the outer contour of the pod nest. In some implementations, the spray-guard is not a sealed device but affords some degree of containment. The spray-guard clips on to the front of the device.

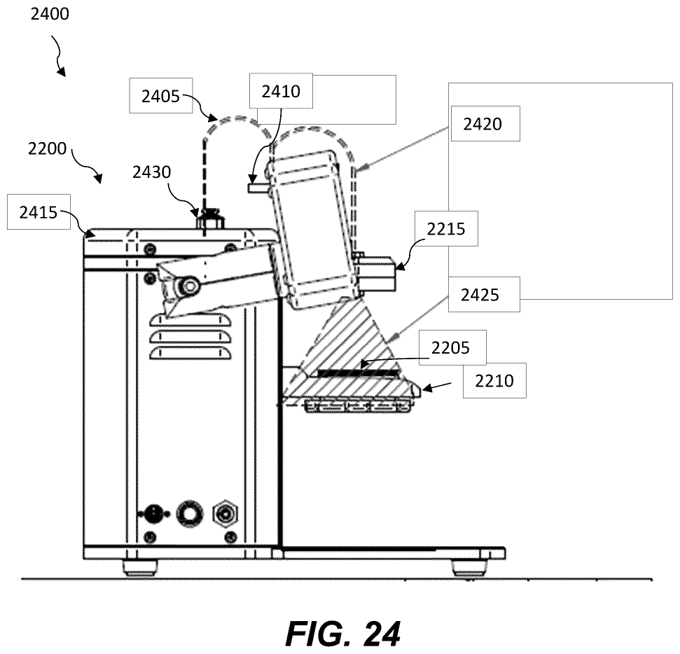

FIG. 24 is a diagram 2400 illustrating a side view of another example embodiment of the CTS 2200 shown in FIG. 22, according to some embodiments disclosed herein. As shown in FIG. 24, the valve can be coupled to the nozzle nest 2215 via one or more portions of tubing. A pneumatic fitting 2430 can include, for example, a Festo 6 mm to 6 mm bulkhead fitting (Catalogue No. 193951). For example, a first portion of tubing 2405 can couple the TBD valve to an Eppendorf base support 2410. The Eppendorf base support 2410 can be coupled to a top cover 2415 of the CTS 2200.

The Eppendorf base support 2410 can include a bracket that holds the payload reservoir in space. An example reservoir includes a 1.5 mL Eppendorf brand centrifuge vial. The reservoir may or may not be permanently fixed in place as the mechanism for securing it to the Eppendorf base support 2410.

A second portion of tubing 2420 can coupled the Eppendorf base support 2410 to the nozzle nest 2215. A permeabilizing solution can be conveyed from a source within the CTS 2200, through the TBD valve and to the Eppendorf base support 2410 via the tubing 2420. The permeabilizing solution can be further provided to the nozzle nest 2215 via tubing 2420. Once received within the nozzle nest 2215, the permeabilizing solution can be provided to the pod 2205 positioned within the pod nest 2210. The nozzle configured within the pod nest 2215 can be configured to deliver the permeabilizing solution to the pod 2205 with a spray pattern 2425. The spray pattern 2425 can be configurable based on a pressure setting at which the permeabilizing solution is provided. In some embodiments, the spray pattern 2425 can be associated with a configuration of a nozzle within the nozzle nest 2215. Dimensions of the spray pattern 2425, such as a spray angle, a coverage area, and/or a center point can be configurable aspects of the nozzle nest 2215.

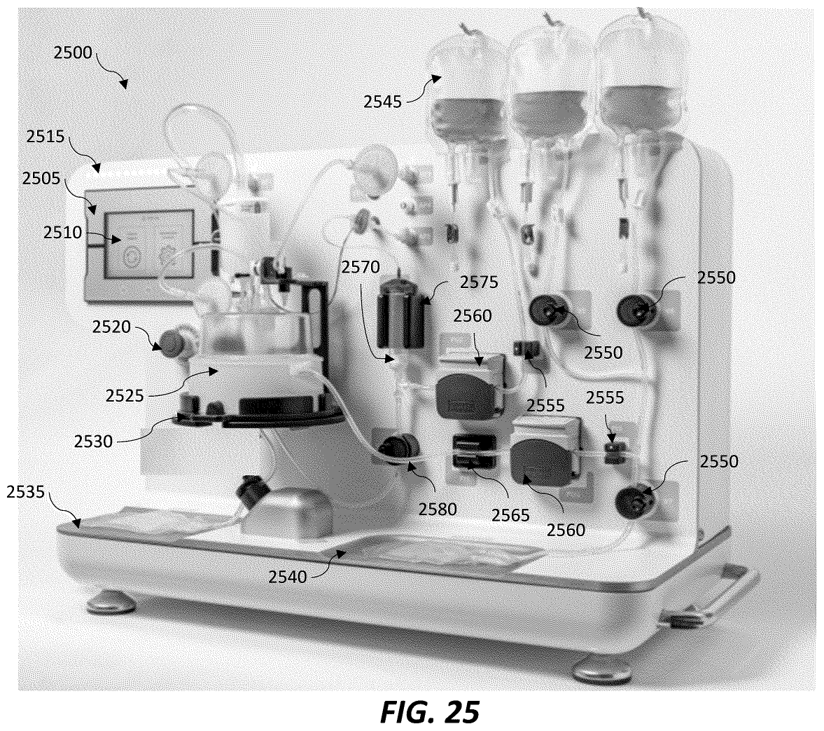

FIG. 25 illustrates an image of another example embodiment of a cell transfection system according to some embodiments disclosed herein. As shown in FIG. 25, in one embodiment, a cell transfection system (CTS) 2500 can include an instrument housing mounted to a base. The base can enable the CTS 2500 to be located or mounted on a bench, within a vented hood workspace, a desktop, a workbench, or the like. In some embodiments, the CTS 2500 can be mounted on a mobile base configured to transport the CTS 2500 from one location to another. In some embodiments, the CTS 2500 can be configured as a closed system for single-use operation to permeabilize cells according to the methods described herein. In some embodiments, the CTS 2500 can be configured to loop through multiple experimental workflows.

As shown in FIG. 25, the CTS 2500 can include a display 2505 providing an HMI 2510. The HMI 2510 can be configured to receive user inputs and to provide outputs associated with operation of the CTS 2500. The HMI 2510 can be electrically coupled to one or more controllers configured within the CTS 2500. The controllers can be configured in a control system of the CTS 2500. The control system can be configured to pump cell suspensions, liquids and air to the nozzle described herein. The control system can be configured to provide spray durations between 100 and 1500 ms at a pressure between 1 and 4 bar. The control system can be configured to reach and to recover from a desired atomization pressure in less than 25 ms. The control system can be further configured to deliver a flow rate of up to 30 L/min. The control system can be configured to deliver a volume of 50 microliters and to control the delivered volume within +/−5 microliters.

The CTS 2500 can also include one or more lights or visual indicators 2515 to indicate one or more statuses associated with operation of the CTS 2500. As shown in FIG. 25, the lights 2515 can include a plurality of lights which can be individually operated with regard to one or more steps or procedures associated with operation of the CTS 2500. In some embodiments, the HMI 2510 can display one or more error or operational codes associated with an operation of the CTS 2500 and the lights 2515 can provide a user with a visual indication corresponding to the codes.

As further shown in FIG. 25, the CTS 2500 can include a stop button 2520. The stop button 2520 can cease operation of the CTS 2500 in the event of user error or operational error when operating the CTS 2500.

As further shown in FIG. 25, the CTS 2500 can include a single-use assembly 2525 mounted within a frame 2530. The single-use assembly 2525 can be provided for use in a sealed, sterile packaging. The single-use assembly 2525 can include a filter upon which cells can be provided for permeabilization and collected following permeabilization. The single-use assembly 2525 can be configured within the frame 2530. In some embodiments, the frame 2530 can be a semi-circular frame or a “C”-shaped frame. The frame 2530 can be mounted to shaft extending from the CTS 2500. The frame 2530, shown in a horizontal orientation in FIG. 25, can be configured to tilt in an upward or downward vertical direction by rotation of the shaft. For example, in some embodiments, the frame 2530 can be configured to tilt 0-10, 5-15, 10-20, 15-25, 20-30, or 25-45 degrees from the horizontal orientation shown in FIG. 25. In some embodiments, the shaft can be configured to tilt the frame 2530 in an oscillating manner with respect to an amount of angular tilt of the frame 2530. For example, the frame 2530 can be tilted to +30 degrees and the frame 2530 can then oscillate between a positive angular orientation (e.g., +1 degree) and a negative angular orientation (e.g., −1 degree) relative to the +30 degree orientation causing the frame 2530 to oscillate between +31 degrees and +29 degrees. The frame 2530 can oscillate between the two angular orientations with a predetermined or user-defined frequency. In some embodiments, the frame 2530 can oscillate at a frequency of 0.5 kHz, 1 kHz, 1.5 kHz, 2 kHz, 2.5 kHz, or more. In some embodiments, the shaft and/or the frame 2530 can be coupled to a servo motor configured to vibrate the shaft and/or the frame 2530.

Oscillating and/or vibrating the frame 2530 can advantageously increase the amount of cells collected following permeabilization compared to aspiration-based collection methods. Aspiration-based collection methods require repeated application and extraction of a collection media within the single-use assembly 2525. In addition, oscillating and/or vibrating the frame 2530 can also advantageously increase the viability of the collected cells, which can be reduced due to exposure to repeated fluid pressures and flow dynamics when cells are collected using aspiration-based collection methods.

As further shown in FIG. 25, the CTS 2500 can include a waste collection tray 2535 at which reagents and/or media evacuated from the single-use assembly 2525 can be collected. In some embodiments, the waste collection tray 2535 can be removed from the CTS 2500. As further shown in FIG. 25, the CTS 2500 can also include a cell collection tray 2540 at which permeabilized cells can be collected. In some embodiments, the cell collection tray 2540 can be removed from the CTS 2500. In some embodiments, the cell collection tray 2540 can include a cooling element and/or a heating element to maintain the permeabilized cells at a desired temperature. In some embodiments, the heating and/or cooling elements associated with the cell collection tray 2540 can be configured within the base of the CTS 2500. In some embodiments, the base can include a scale located underneath the waste collection tray 2535 and/or the cell collection tray 2540. In this way, the CTS 2500 can determine a weight of collected media materials and collected cells.

As further shown in FIG. 25, the CTS 2500 can include one or more media materials 2545. The media materials 2545 can be fluidically coupled to the chamber 2530 via one or more fluid circuits. The CTS 2500 can also include one or more valves 2550 configured to control an amount of media provided via the one or more fluid circuits. In some embodiments, the one or more values 2550 can include pinch valves. The CTS can include one or more fluid detection sensors 2555 configured in-line with respect to a corresponding fluid circuit. The fluid detection sensors 2555 can be configured to aid priming as well as calibration of the CTS 2500. In addition, the fluid detection sensors 2555 can be configured as a measurement system to calculate a volume of media circuit between two locations. As further shown in FIG. 25, the CTS 2500 can include one or more pumps 2560 to pump cell culture media into the single-use assembly 2525. By pumping cell culture media into and out of the single-use assembly 2525 in a cyclic manner, while the frame 2530 is being vibrated and/or tilted, cell collection can be increased compared to non-tilting, non-vibrating cell collection operations. In some embodiments, the pumps 2560 can include peristaltic pumps. Other example pump types can include syringe pump, plunger-less syringe pump, closed syringe types, bag squeezer pump, and the like. The CTS 2500 can also include an ultrasonic flow rate detector 2565.

As further shown in FIG. 25, the CTS 2500 can include a syringe 2570. In some embodiments, the syringe 2570 can include a plunger-less syringe. Air can be applied to the syringe 2570 to provide the media 2545 to the single-use assembly 2525. The CTS 2500 can also include an optical detector 2575 configured within a holder of the syringe 2570 or within the CTS 2500 itself. The optical detector 2575 can detect a level of fluid within the syringe 2570 or the position of a plunger or a bung of the syringe 2570. The optical detector 2575 can include an array of optical sensors, such as infra-red detectors, arranged linearly in a vertical array. The optical detector 2575 can be used in calibration operations in combination with the pump 2560. In some embodiments, the syringe 2570 can be coupled to a check valve located at an exit of the syringe 2570. The fluid detector 2575 can be coupled to a valve 2580 to control an amount of media provided to the single-use assembly 2525.

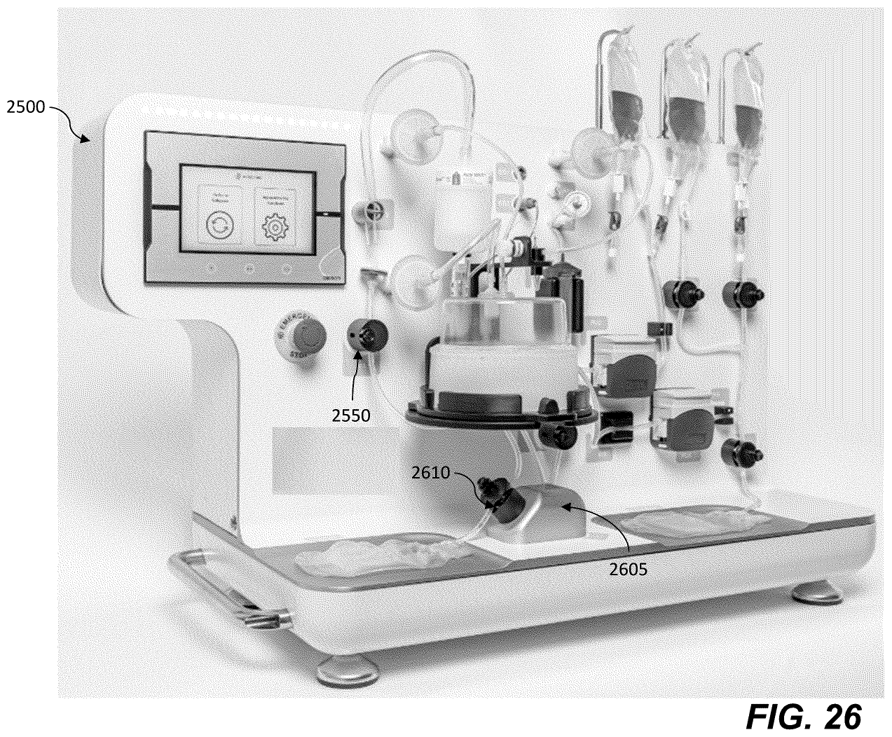

FIG. 26 illustrates a view of the cell transfection system 2500 shown in FIG. 25. As shown in FIG. 26, the CTS 2500 can include a valve holder 2605 configured to hold a valve 2610. The valve holder 2605 can be configured to hold the valve 2610 at an angle relative to an orientation of the single-use assembly 2525.

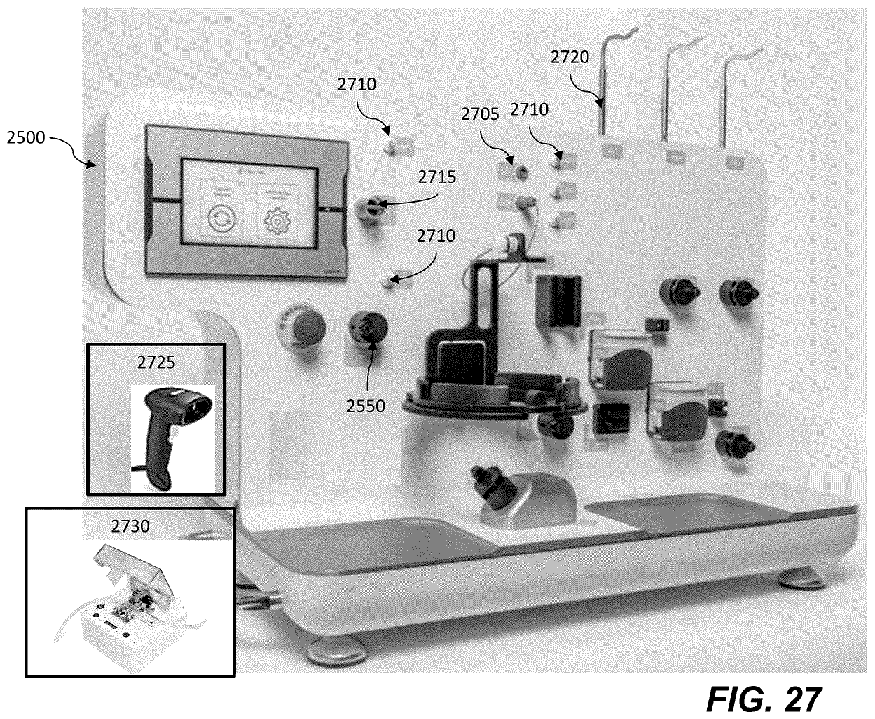

FIG. 27 illustrates a second view of the cell transfection system shown in FIG. 25. As shown in FIG. 27, the CTS 2500 can include one or more electrical connectors 2705. In some embodiments, the electrical connectors 2705 can be instrument connectors to connect external instrumentation equipment to the CTS 2500. The external instrumentation can include, for example, an electrical thermometer, a hydrometer, a barometer, photoplethysmograph sensor, load cells, biochemical sensor (e.g., an alcohol sensor), optical sensor, transducer to measure vibration (e.g., vibrating membrane microelectronic machine (MEMs)), and the like.

The CTS 2500 can also include one or more gas connectors 2710. The gas connectors 2710 can receive a gas supply and provide the gas supply to the single-use assembly 2525 under desired pressure conditions. In some embodiments, the gas connectors 2710 can receive a gas from the single-use assembly 2525, for example when purging or venting the single-use assembly 2525. In some implementations, the gas connectors 2710 can be independently controlled via software, and each gas connector 2710 can be configured to provide a static or dynamic head of pressure (e.g., a pressure set point). In some implementations, the gas connectors 2710 can operate with different gases (e.g., medical, nitrogen, and the like), can be software configurable, can provide contiguous airflow at a specified pressure into the vessel, and the like. Pressure can be provided by a flow control regulator, pressure regulator, flow transducer, pressure transducer, and the like. Pressure can be provided to other components such as a nozzle, a shower head, an Eppendorf needle, to drive the bung in a plunger-less syringe, and the like. Each gas connector 2710 can be can be independently software configurable and not part of a manifold.

Valve 2550 can be coupled to a fluid circuit associated with one of the gas connectors 2710 and can control an amount of gas supplied to the single-use assembly 2525. The CTS 2500 can include a hose clamp 2715 to secure a portion of hose configured for use with the single-use assembly 2525. The CTS 2500 can also include one or more hangers 2720 to hold media 2545. In some embodiments, the hangers 2720 can be configured with scales to determine a weight of the media 2545.

As shown in FIG. 27, the CTS 2500 can include a bar code reader 2725. The bar code reader 2725 can be configured to scan bar-coded media, a badge associated with an operator of the CTS 2500, and/or bar-coded packaging containing the single-use assembly 2525. In some embodiments, the bar code reader 2725 can include a linear bar code reader or 2-D bar code reader. In some embodiments, the bar code reader 2725 can be a hand-held bar code reader. In some embodiments, the HMI 2510 can be communicatively coupled to the bar code reader 2725. Additionally, the CTS 2500 can include a tube welder 2730 configured to fix or apply a weld to tubing of the CTS 2500, such as tubing used in association with the one or more fluid circuits coupled to the media 2545. In some embodiments, the HMI 2510 can be communicatively coupled to the tube welder 2730.

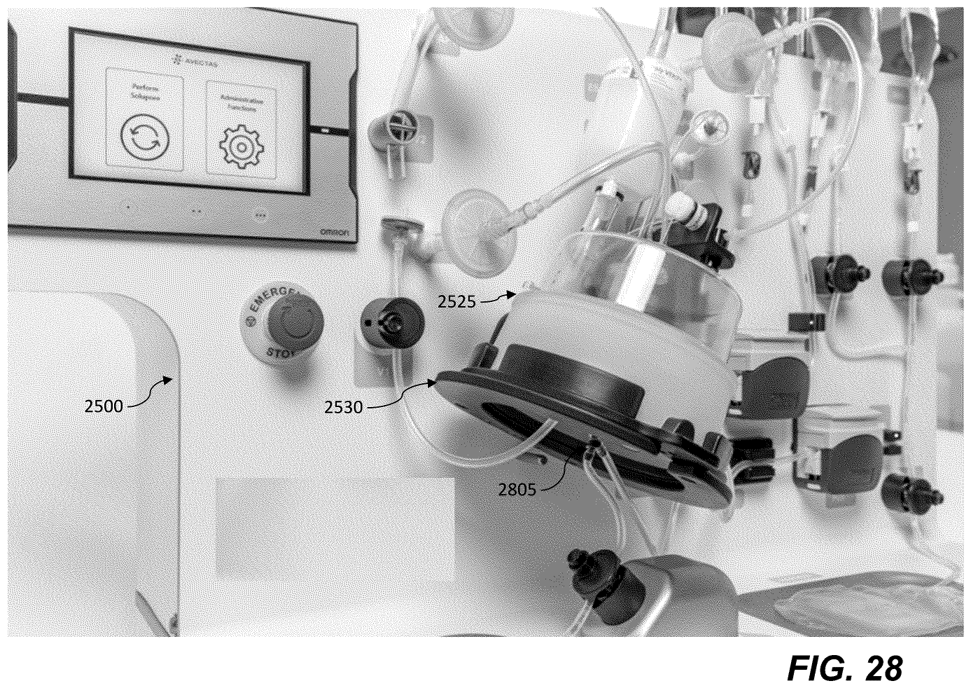

FIG. 28 illustrates a close-up view of a portion of the cell transfection system shown in FIG. 25. As shown in FIG. 25, the single-use assembly 2525 and the frame 2530 are tilted at about a +30 degree angular orientation relative to the horizontal orientation shown in FIG. 25. In this position, the frame 2530 can oscillate in positive and negative angular movements from the +30 degree angular orientation to aid collection of cells via a drain 2805 configured in the bottom of the single-use assembly 2525.

### Other Embodiments

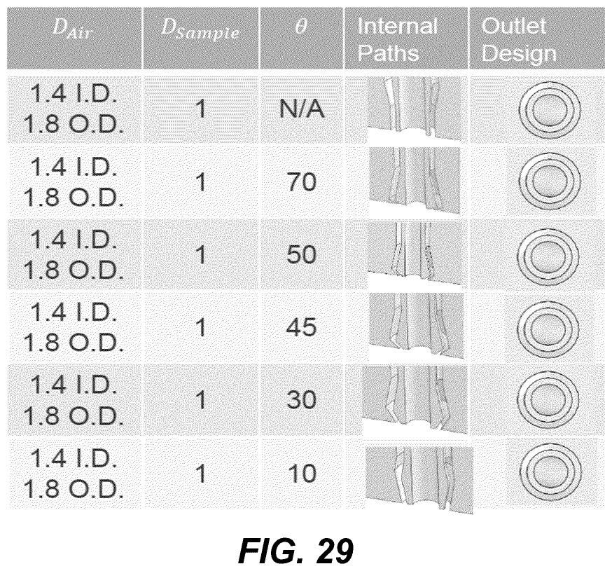

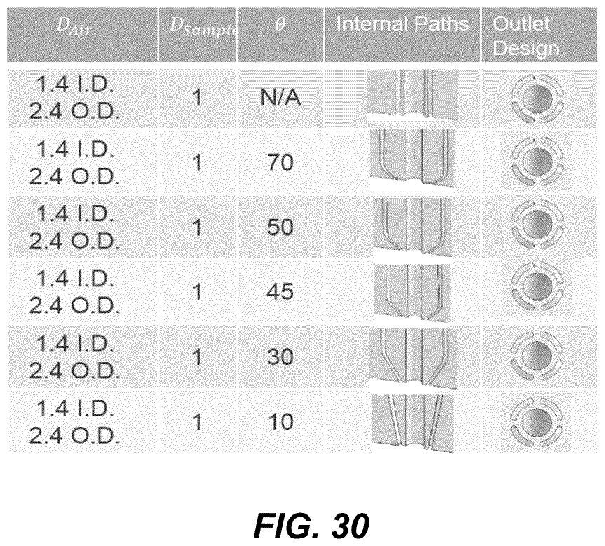

FIG. 29 is a table showing additional embodiments of an implementation of the nozzle of FIGS. 1A-1D. The features of the embodiments shown in the table can correspond to the features of the embodiments of the nozzles 100 and 600 shown and described in relation to FIGS. 1A-1D and 6A-8B. In the embodiments shown in FIG. 29, a single annularly-shaped gas delivery passage is arranged around a single sample delivery passage. As shown in the table of FIG. 30, the gas delivery passage (DAir) can have an inner dimeter (I.D.) of 1.4 mm and an outer diameter (O.D.) of 1.8 mm. The diameter of the sample delivery passage (DSample) can be 1 mm. The taper angle, θ, can be between 0-70 degrees. As shown in the column labeled “Internal Paths”, the gas delivery passages can have a shape based on the corresponding taper angle, θ. The taper angle, θ can allow for variable relative speeds between the sample outlet 150 and the gas outlet 145. A theta=0 degrees can maximize the relative speed. The column labeled “Outlet Design” can correspond to the shape and arrangement of the sample outlet and the gas outlet at the output orifice of the nozzle.

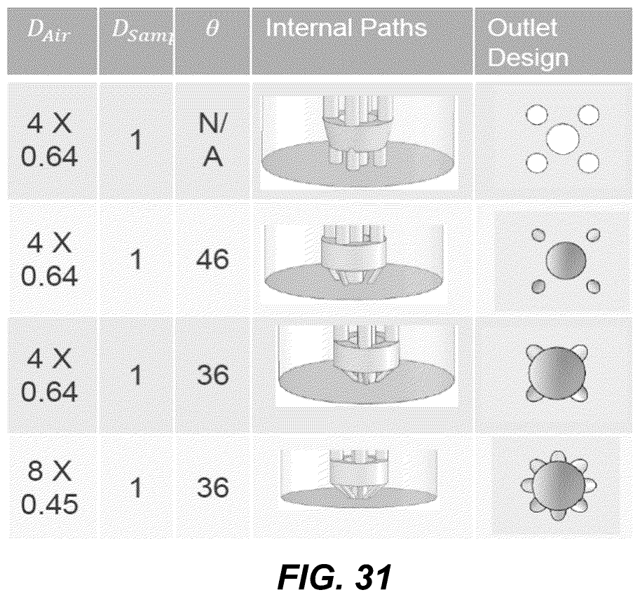

FIG. 30 is a table showing additional embodiments of another implementation of the nozzle of FIGS. 1A-1D. The features of the embodiments shown in the table can correspond to the features of the embodiments of the nozzles 100 and 600 shown and described in relation to FIGS. 1A-1D and 6A-8B. In the embodiments shown in FIG. 30, a multiple semi-circular-shaped gas delivery passages are arranged around a single sample delivery passage. As shown in the table of FIG. 31, the gas delivery passage (DAir) can have an inner dimeter (I.D.) of 1.4 mm and an outer diameter (O.D.) of 2.4 mm. The diameter of the sample delivery passage (DSample) can be 1 mm. The taper angle, θ, can be between 0-70 degrees. As shown in the column labeled “Internal Paths”, the gas delivery passages can have a shape based on the corresponding taper angle, θ. The taper angle, θ can allow for variable relative speeds between the sample outlet 150 and the gas outlet 145. A theta=0 degrees can maximize the relative speed. The column labeled “Outlet Design” can correspond to the shape and arrangement of the sample outlet and the multiple gas outlets at the output orifice of the nozzle.

FIG. 31 is a table showing additional embodiments of an implementation of the nozzle of FIGS. 9A-9D. The features of the embodiments shown in the table can correspond to the features of the embodiments of the nozzles 900 and 1400 shown and described in relation to FIGS. 9A-9D and 14A-14C. In the embodiments shown in FIG. 31, a multiple circular or semi-circular-shaped gas delivery passages are arranged around a single sample delivery passage. As shown in the table of FIG. 32, in some embodiments, the nozzle can include 4 or 8 gas delivery passages arranged around the circumference of the sample delivery passage. The gas delivery passages (DAir) can have a dimeter of 0.64 mm or 0.45 mm. The diameter of the sample delivery passage (DSample) can be 1 mm. As shown in the column labeled “Internal Paths”, the output passages exiting the cylindrically-shaped mixing chamber at which the can be formed at one or more output passage angles, θ. The output passage angle θ can be between 0-46 degrees. The output passage angle θ can allow for variable relative speeds between the mixing chamber 955, the output passage 960, or gas output passages which can each be fluidically coupled to the mixing chamber as shown in the embodiments of FIG. 31. A theta=0 degrees can maximize the relative speed. The column labeled “Outlet Design” can correspond to the shape and arrangement of the sample outlet, the gas outlet(s), and the output passage at the output orifice of the nozzle.

Nozzle 100 and nozzle 900 described herein can enable atomization of samples using pulse of sprays or a pulsatile spray. Nozzle 100 and nozzle 900 can provide “fast on” and “fast off” operation. The nozzle 100 and nozzle 900 do not require additional time to form a steady-state consistent spray as compared to existing nozzles, such as those found in metered dose inhalers. Additionally, the nozzle 100 and the nozzle 900 described herein can provide the consistent spray without producing drops of sample and without operating intermittently to form spurts of sample. At the exact moment the nozzle 100 or the nozzle 900 is in use, the design of the nozzles allows for minimal to no ramp up time compared to existing nozzle designs. Further the rapid and consistent “fast on” and “fast off” operation can be advantageous for pulse delivery of a sample, such as on/off/on/off sequencing. While embodiments of the nozzles described herein are provided with respect to current contemplation is to single sprays of cells with a sample or a cargo, the nozzles described herein can advantageously improve operation using pulses or pulsed delivery. While temporal and spatial delivery of cargo reaching a cell via a pulsatility inherently found within a spray plume, the described nozzle can be used in a directed and refined pulse transfection workflow. For example, in a cell transfection workflow, the spray from nozzles 100 or 900 can provide a “fast on” spray of 10-100 ms, followed by a “fast off” and further performing 1, 2, 3, 4, 5, or more on/off intervals to deliver samples or cargo to the cells before performing a final off spray. The delivery of cargo, also referred to as cargo dosing, can be advantageously performed using the nozzle 100 and nozzle 900 described herein based on effectively controlling the spray volume and the spray time via the back pressure of the nozzle design described in relation to FIG. 8.

In the descriptions above and in the claims, phrases such as “at least one of” or “one or more of”' may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” In addition, use of the term “based on,” above and in the claims is intended to mean, “based at least in part on,” such that an unrecited feature or element is also permissible.

### Exemplary Embodiments

Embodiment 1 is a cell-transfection spray nozzle apparatus for delivering biologically compatible aqueous-based compositions onto cells includes:
a needle comprising:
   a hub having a sample inlet configured for receiving a liquid sample;
   a proximal end portion coupled to the hub and defining a first inner diameter extending along a central axis; and
   a distal end portion connected to the proximal end portion and having a sample outlet for dispensing the liquid sample, the distal end portion defining a second inner diameter that is larger than the first inner diameter;
a sleeve comprising:
   a body extending from a proximal end to a distal end, the distal end comprising a distal tip, the body defined by exterior walls and interior walls, at least a portion of the interior and exterior walls extending at an angle relative to the central axis between the proximal and distal ends, the interior walls at the proximal and distal ends being dimensioned to receive the hub and distal end portion of the needle, respectively; and
   four wings radially spaced from one another and extending from at least a portion of the angled exterior walls of the body;
a housing configured to receive the sleeve, the housing comprising:
   an air inlet portion having an air inlet configured for receiving air;
   a first cylindrical portion fluidically coupled to the air inlet portion, the first cylindrical portion configured to releasably couple with the proximal end of the sleeve;
   a second cylindrical portion having an inner cylindrical diameter that is smaller than that of the first cylindrical portion, the second cylindrical portion configured to receive the distal end of the sleeve;
   a first conical portion fluidically coupled to and extending between the first and second cylindrical portions;
   a second conical portion coupled to the second cylindrical portion and having an air outlet configured for dispensing the air, the second conical portion defined by a maximum inner diameter that is equal to a minimum inner diameter of the first conical portion; and
wherein the needle, the sleeve, and the housing together define cavities configured for flowing the air, the cavities comprising:
   a first cavity comprising the air inlet portion of the housing and an annular space defined by at least portion of the first cylindrical portion of the housing, the first conical portion of the housing, and the exterior walls of the sleeve;
   a plurality of second cavities that are adjacent the first cavity and defined by at least a portion of the second cylindrical portion of the housing, the wings and the angled exterior walls of the body of the sleeve;
   a third cavity fluidically connected to the first cavity via the plurality of the second cavities, the third cavity extending distally in a direction along the central axis by a predetermined length that is distal to the distal tip of the sleeve; the third cavity defined by at least a portion of the exterior walls of the sleeve, the second conical portion of the housing and distal end of the needle; and
   the second cavities defining a volume that is smaller than that of the first cavity, the third cavity, or both.

Embodiment 2 is a spray nozzle apparatus of embodiment 1, wherein the sample outlet is concentrically positioned within the air outlet.

Embodiment 3 is a spray nozzle apparatus of embodiment 1 or embodiment 2, wherein the four wings are equidistant from one another.

Embodiment 4 is a spray nozzle apparatus of any one of embodiments 1-3, wherein the needle expands to the second diameter proximal to the first conical portion.

Embodiment 5 is a spray nozzle apparatus of any one of embodiments 1-4, wherein the first cylindrical portion extends in a direction along the central axis, and wherein the air inlet is arranged at a right angle with respect to the first cylindrical portion.

Embodiment 6 is a spray nozzle apparatus of any one of embodiments 1-5, wherein the internal walls of the first and second conical portions are tapered at a same taper angle.

Embodiment 7 is a spray nozzle apparatus of any one of embodiments 1-6, wherein the plurality of second cavities are coplanar to one another.

Embodiment 8 is a spray nozzle apparatus of any one of embodiments 1-7, wherein the first diameter of the needle is at least 20% smaller than the second diameter of the needle.

Embodiment 9 is a spray nozzle apparatus of any one of embodiments 1-8, wherein the sleeve includes four wings spaced apart equidistant around the sleeve, the four wings are tapered to fit within the second cylindrical portion.

Embodiment 10 is a spray nozzle apparatus of any one of embodiments 1-9, wherein sample droplets are sprayed from the nozzle apparatus such that the sample droplets have a droplet size of about 10 to about 10.9 μm, about 11 to about 11.9 μm, about 12 to about 12.9 μm, about 13 to about 13.9 μm, about 14 to about 14.9 μm, about 15 to about 15.9 μm, about 16 to about 16.9 μm, or about 17 to about 18 μm; and wherein 80% or more of the sample droplets produced at the sample outlet have the droplet size.

Embodiment 11 is a method of delivering atomized fluids onto cells using a cell transfection nozzle apparatus, the method comprising:
introducing the liquid sample to the sample inlet of the nozzle apparatus of embodiment 1,
introducing the air from a gas source to the gas inlet of the nozzle apparatus;
flowing the gas through the cavities of the nozzle apparatus; and
dispensing an atomized spray of sample droplets formed by the liquid sample exiting from the sample outlet and the gas exiting from the gas outlet of the housing.

Embodiment 12 is a method of embodiment 11, wherein the dispensing comprises shearing the liquid sample exiting from the sample outlet with the gas exiting from the gas outlet.

Embodiment 13 is a method of embodiment 11 or embodiment 12, wherein the flowing the gas through the cavities of the nozzle apparatus comprises:
flowing the gas through the first cavity;
flowing the gas through the plurality of second cavities;
flowing the gas through the third cavity fluidically connected to the first cavity via the plurality of the second cavities.

Embodiment 14 is a method of any one of embodiments 11-13, wherein the flowing the gas through the cavities of the nozzle apparatus provides a laminar flow of gas at the third cavity, at the gas outlet, or both.

Embodiment 15 is a method of any one of embodiments 11-14, wherein the first cavity subdivides the gas flowing from the gas inlet portion of the housing.

Embodiment 16 is a method of any one of embodiments 11-15, comprising subdividing the gas into four separate passages via the plurality of second cavities that are located between the first cavity and the third cavity of the nozzle apparatus.

Embodiment 17 is a method of embodiment 15 or embodiment 16, comprising recombining the subdivided gas in the third cavity.

Embodiment 18 is a method of any one of embodiments 11-17, comprising spraying the sample droplets from the nozzle apparatus such that the sample droplets have a droplet size of about 10 to about 10.9 μm, about 11 to about 11.9 μm, about 12 to about 12.9 μm, about 13 to about 13.9 μm, about 14 to about 14.9 μm, about 15 to about 15.9 μm, about 16 to about 16.9 μm, or about 17 to about 18 μm; and wherein 80% or more of the sample droplets produced at the sample outlet have the droplet size.

Embodiment 19 is an apparatus comprising:
a housing including a first end and a second end, the first end including a sample inlet and a gas inlet, and the second end including a sample outlet and a gas outlet;
a sample delivery passage extending within the housing and fluidically coupling the sample inlet to the sample outlet; and
a gas delivery passage extending within the housing and fluidically coupling the gas inlet to the gas outlet.

Embodiment 20 is a method comprising:
coupling a sample source including a sample to a sample inlet arranged at an first end of a housing of a nozzle;
coupling a gas source including a gas to a gas inlet arranged at the first end of the housing of the nozzle;
providing the sample within a sample delivery passage extending within the housing and fluidically coupling the sample inlet to a sample outlet arranged at an output orifice at a second end of the housing of the nozzle; and
providing the gas within a gas delivery passage extending within the housing and fluidically coupling the gas inlet to a gas outlet arranged at the output orifice at the second end of the housing of the nozzle; wherein the gas and the sample are dispensed from the output orifice as an atomized spray to cells arranged on a filter membrane of a cell transfection system.

Embodiment 21 is a nozzle apparatus comprising a needle, a sleeve, and a housing configured to receive the sleeve. The needle includes a hub having a sample inlet configured for receiving a liquid sample; a proximal end portion coupled to the hub and defining a first inner diameter extending along a central axis; and a distal end portion connected to the proximal end portion and having a sample outlet for dispensing the liquid sample. The sleeve includes a body extending from a proximal end to a distal end, the distal end comprising a distal tip, the body defined by exterior walls and interior walls; and a plurality of wings radially spaced from one another and extending from the exterior walls of the body. The housing includes a gas inlet portion having a gas inlet configured for receiving a gas; a first portion fluidically coupled to the gas inlet portion; a second portion configured to receive the distal end of the sleeve; a third portion that is a conical in shape and fluidically coupled to and extending between the first and second portions; and a fourth portion that is conical in shape and coupled to the second portion and having a gas outlet configured for dispensing the gas. The housing, the sleeve, and needle together define cavities configured for flowing the gas, the cavities including: a first cavity comprising the gas inlet portion and an annular space defined by at least a portion of the first and third portions of the housing and the exterior walls of the sleeve; a plurality of second cavities that are adjacent the first cavity and defined by at least a portion of the second portion of the housing, the wings of the sleeve, and the exterior walls of the body of the sleeve; and a third cavity fluidically connected to the first cavity via the plurality of the second cavities, wherein the third cavity extends distally in a direction along the central axis by a predetermined length that is distal to the distal tip of the sleeve; and the third cavity is defined by at least a portion of the exterior walls of the sleeve, the third portion of the housing and distal end of the needle.

Embodiment 22 is a nozzle apparatus comprising a needle, a sleeve, and a housing configured to receive the sleeve. The needle includes a hub having a sample inlet configured for receiving a liquid sample; a proximal end portion coupled to the hub and defining a first inner diameter extending along a central axis; and a distal end portion connected to the proximal end portion and having a sample outlet for dispensing the liquid sample. The sleeve includes a body extending from a proximal end to a distal end, the distal end comprising a distal tip, the body defined by exterior walls and interior walls; and a plurality of wings radially spaced from one another and extending from the exterior walls of the body. The housing includes a gas inlet portion having a gas inlet configured for receiving a gas; a first portion fluidically coupled to the gas inlet portion; a second portion configured to receive the distal end of the sleeve; a third portion that is fluidically coupled to and extending between the first and second portions; and a fourth portion that is coupled to the second portion and having a gas outlet configured for dispensing the gas. The housing, the sleeve, and needle together define cavities configured for flowing the gas, the cavities including: a first cavity comprising the gas inlet portion and an annular space defined by at least a portion of the first and third portions of the housing and the exterior walls of the sleeve; a plurality of second cavities that are adjacent the first cavity and defined by at least a portion of the second portion of the housing, the wings of the sleeve, and the exterior walls of the body of the sleeve; and a third cavity fluidically connected to the first cavity via the plurality of the second cavities.

While this specification contains many specific implementation details, these should not be construed as limitations on the scope of the disclosed technology or of what may be claimed, but rather as descriptions of features that may be specific to particular embodiments of particular disclosed technologies. Certain features that are described in this specification in the context of separate embodiments can also be implemented in combination in a single embodiment in part or in whole. Conversely, various features that are described in the context of a single embodiment can also be implemented in multiple embodiments separately or in any suitable subcombination. Moreover, although features may be described herein as acting in certain combinations and/or initially claimed as such, one or more features from a claimed combination can in some cases be excised from the combination, and the claimed combination may be directed to a subcombination or variation of a subcombination. Similarly, while operations may be described in a particular order, this should not be understood as requiring that such operations be performed in the particular order or in sequential order, or that all operations be performed, to achieve desirable results. Particular embodiments of the subject matter have been described. Other embodiments are within the scope of the following claims.

Accordingly, other implementations are within the scope of the following claims.

What is claimed is:

1. A cell-transfection spray nozzle apparatus for delivering biologically compatible aqueous-based compositions onto cells, the nozzle apparatus comprising:
a needle comprising:
a hub having a sample inlet configured for receiving a liquid sample;
a proximal end portion coupled to the hub and defining a first inner diameter extending along a central axis; and
a distal end portion connected to the proximal end portion and having a sample outlet for dispensing the liquid sample, the distal end portion defining a second inner diameter that is larger than the first inner diameter;
a sleeve comprising:
a body extending from a proximal end to a distal end, the distal end comprising a distal tip, the body defined by exterior walls and interior walls, at least a portion of the interior and exterior walls extending at an angle relative to the central axis between the proximal and distal ends, the interior walls at the proximal and distal ends being dimensioned to receive the hub and distal end portion of the needle, respectively; and
four wings radially spaced from one another and extending from at least a portion of the angled exterior walls of the body;
a housing configured to receive the sleeve, the housing comprising:
an air inlet portion having an air inlet configured for receiving air;
a first cylindrical portion fluidically coupled to the air inlet portion, the first cylindrical portion configured to releasably couple with the proximal end of the sleeve;
a second cylindrical portion having an inner cylindrical diameter that is smaller than that of the first cylindrical portion, the second cylindrical portion configured to receive the distal end of the sleeve;
a first conical portion fluidically coupled to and extending between the first and second cylindrical portions;
a second conical portion coupled to the second cylindrical portion and having an air outlet configured for dispensing the air, the second conical portion defined by a maximum inner diameter that is equal to a minimum inner diameter of the first conical portion; and
wherein the needle, the sleeve, and the housing together define cavities configured for flowing the air, the cavities comprising:
a first cavity comprising the air inlet portion of the housing and an annular space defined by at least portion of the first cylindrical portion of the housing, the first conical portion of the housing, and the exterior walls of the sleeve;
a plurality of second cavities that are adjacent the first cavity and defined by at least a portion of the second cylindrical portion of the housing, the wings and the angled exterior walls of the body of the sleeve;
a third cavity fluidically connected to the first cavity via the plurality of the second cavities, the third cavity extending distally in a direction along the central axis by a predetermined length that is distal to the distal tip of the sleeve; the third cavity defined by at least a portion of the exterior walls of the sleeve, the second conical portion of the housing and distal end of the needle; and
the second cavities defining a volume that is smaller than that of the first cavity, the third cavity, or both.

2. The spray nozzle apparatus of claim 1, wherein the sample outlet is concentrically positioned within the air outlet.

3. The spray nozzle apparatus of claim 1, wherein the four wings are equidistant from one another.

4. The spray nozzle apparatus of claim 1, wherein the needle expands to the second diameter proximal to the first conical portion.

5. The spray nozzle apparatus of claim 1, wherein the first cylindrical portion extends in a direction along the central axis, and wherein the air inlet is arranged at a right angle with respect to the first cylindrical portion.

6. The spray nozzle apparatus of claim 1, wherein the internal walls of the first and second conical portions are tapered at a same taper angle.

7. The spray nozzle apparatus of claim 1, wherein the plurality of second cavities are coplanar to one another.

8. The spray nozzle apparatus of claim 1, wherein the first diameter of the needle is at least 20% smaller than the second diameter of the needle.

9. The spray nozzle apparatus of claim 1, wherein the sleeve includes four wings spaced apart equidistant around the sleeve, the four wings are tapered to fit within the second cylindrical portion.

10. The spray nozzle apparatus of claim 1, wherein sample droplets are sprayed from the nozzle apparatus such that the sample droplets have a droplet size of about 10 to about 10.9 μm, about 11 to about 11.9 μm, about 12 to about 12.9 μm, about 13 to about 13.9 μm, about 14 to about 14.9 μm, about 15 to about 15.9 μm, about 16 to about 16.9 μm, or about 17 to about 18 μm; and wherein 80% or more of the sample droplets produced at the sample outlet have the droplet size.

11. A method of delivering atomized fluids onto cells using a cell transfection nozzle apparatus, the method comprising:
introducing the liquid sample to the sample inlet of the nozzle apparatus of claim 1, introducing the air from a gas source to the gas inlet of the nozzle apparatus;
flowing the gas through the cavities of the nozzle apparatus; and
dispensing an atomized spray of sample droplets formed by the liquid sample exiting from the sample outlet and the gas exiting from the gas outlet of the housing.

12. The method of claim 11, wherein the dispensing comprises shearing the liquid sample exiting from the sample outlet with the gas exiting from the gas outlet.

13. The method of claim 12, wherein the flowing the gas through the cavities of the nozzle apparatus comprises:
flowing the gas through the first cavity;
flowing the gas through the plurality of second cavities;
flowing the gas through the third cavity fluidically connected to the first cavity via the plurality of the second cavities.

14. The method of claim 13, wherein the flowing the gas through the cavities of the nozzle apparatus provides a laminar flow of gas at the third cavity, at the gas outlet, or both.

15. The method of claim 13, wherein the first cavity subdivides the gas flowing from the gas inlet portion of the housing.

16. The method of claim 13, comprising subdividing the gas into four separate passages via the plurality of second cavities that are located between the first cavity and the third cavity of the nozzle apparatus.

17. The method of claim 16, comprising recombining the subdivided gas in the third cavity.

18. The method of claim 11, comprising spraying the sample droplets from the nozzle apparatus such that the sample droplets have a droplet size of about 10 to about 10.9 μm, about 11 to about 11.9 μm, about 12 to about 12.9 μm, about 13 to about 13.9 μm, about 14 to about 14.9 μm, about 15 to about 15.9 μm, about 16 to about 16.9 μm, or about 17 to about 18 μm; and wherein 80% or more of the sample droplets produced at the sample outlet have the droplet size.

19. An apparatus comprising:
a housing including a first end and a second end, the first end including a sample inlet and a gas inlet, and the second end including a sample outlet and a gas outlet;
a sample delivery passage extending within the housing and fluidically coupling the sample inlet to the sample outlet; and
a gas delivery passage extending within the housing and fluidically coupling the gas inlet to the gas outlet.

20. A method comprising:
coupling a sample source including a sample to a sample inlet arranged at an first end of a housing of a nozzle;
coupling a gas source including a gas to a gas inlet arranged at the first end of the housing of the nozzle;
providing the sample within a sample delivery passage extending within the housing and fluidically coupling the sample inlet to a sample outlet arranged at an output orifice at a second end of the housing of the nozzle; and
providing the gas within a gas delivery passage extending within the housing and fluidically coupling the gas inlet to a gas outlet arranged at the output orifice at the second end of the housing of the nozzle; wherein the gas and the sample are dispensed from the output orifice as an atomized spray to cells arranged on a filter membrane of a cell transfection system.
